(19) United States
(12) Patent Application Publication
(10) Pub. No.: US 2024/0241137 A1
(43) Pub. Date: Jul. 18, 2024

(54) METHOD FOR SELECTING A DRUG CANDIDATE FOR TREATING OR PREVENTING OR DELAYING THE ONSET OR PROGRESSION OF AN AMYLOIDOGENIC DISEASE

(71) Applicant: NATIONAL HEALTH RESEARCH INSTITUTES, Miaoli County (TW)

(72) Inventor: Shu-Yi LIN, Miaoli County (TW)

(21) Appl. No.: 18/543,232

(22) Filed: Dec. 18, 2023

Related U.S. Application Data
(60) Provisional application No. 63/476,493, filed on Dec. 21, 2022.

(51) Int. Cl. G01N 33/68; G01N 33/92

(52) U.S. Cl. CPC G01N 33/6896; G01N 33/92; G01N 2500/20; G01N 2800/52

(57) ABSTRACT

The present disclosure provides a method for selecting a drug candidate for treating or preventing or delaying the onset or progression of an amyloidogenic disease, comprising: (a) contacting an analyte with amyloid peptide 1-42 (Aβ42) and an amphiphilic liposaccharide or a part thereof, wherein the amphiphilic liposaccharide or the part thereof is capable of forming a complex with Aβ42; and (b) determining if the analyte extends an oscillatory event of the complex; and the analyte is selected as the drug candidate.

### CROSS-REFERENCE TO RELATED APPLICATIONS

This application claims priority to U.S. Provisional Patent Application No. 63/476,493, filed Dec. 21, 2022, which is entirely incorporated herein by reference.

### FIELD OF THE INVENTION

The present disclosure relates to a method for selecting a drug candidate, particularly to a method for selecting a drug candidate for treating or preventing or delaying the onset or progression of an amyloidogenic disease.

### BACKGROUND OF THE INVENTION

Amyloid peptide 1-42 (Aβ42), also referred to as Aβ(1-42), is a family of proteins ranging from 1-39, 1-40, 1-41, 1-42 and 1-43 residues in length. The 1-42 form is a central component of insoluble extracellular depositions (senile or neuritic plaques) composed of proteins, lipids, carbohydrates and salts in the brains of patients with Alzheimer's or Down's syndrome. Amyloid peptides (Aβ) undergo self-aggregation to form amyloid plaques in triggering the neurodegenerative cascade of AD. Among the various Aβ isoforms, Aβ40 and Aβ42 are the two most abundant species. Although Aβ40 is more abundant than Aβ42 in cerebrospinal fluid, Aβ42 has a higher self-aggregation potential than Aβ40 in contributing to the amyloid deposits in AD brains. From the perspective of chemical kinetics, the self-propagation of Aβ42 represents a dynamic self-assembly process involving a transition state from the pre-organized protofibrils to a stable form of fibrils.

Altered homeostasis between amyloid peptide 1-42 (Aβ42) production and clearance is defined as the pathological basis for the accumulated Aβ fibrils in Alzheimer's disease (AD) brains. However, efforts aimed at blocking Aβ42 production have not been successful, as evidenced by the fact that more than 200 clinical trials of drugs designed to decrease Aβ42 production have been terminated. There is thus a need in the art for treatment of amyloidogenic diseases.

### SUMMARY OF THE INVENTION

The present disclosure provides a method for selecting a drug candidate for treating or preventing or delaying the onset or progression of an amyloidogenic disease, comprising:
(a) contacting an analyte with Aβ42 and an amphiphilic liposaccharide or a part thereof, wherein the amphiphilic liposaccharide or the part thereof is capable of forming a complex with Aβ42; and
(b) determining if the analyte extends an oscillatory event of the complex; and the analyte is selected as the drug candidate.

In some embodiments, the drug candidate is for clearance of Aβ42.

In some embodiments, the amphiphilic liposaccharide comprises a lipid A part and an O-antigen part. In some embodiments, the amphiphilic liposaccharide comprises one or more of lipopolysaccharide (LPS), monophosphoryl lipid A (MPL), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-0-phosphono-α-D-glucopyranose (PIX), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose (PXI) and salts thereof.

In some embodiments, step (a) comprises contacting the analyte with Aβ42 and the lipid A part or the O-antigen part of the amphiphilic liposaccharide.

In some embodiments, step (b) comprises observing the oscillatory events of the complex in the absence and presence of the analyte. In some embodiments, step (b) comprises analyzing variation of mass signals over time to observe the oscillatory event of the complex. In some embodiments, step (b) further comprises acquiring frequency signals and converting the frequency signals to the mass signals.

In some embodiments, step (b) comprises:
(b1) obtaining a first lifetime of the oscillatory event of the complex in the absence of the analyte;
(b2) obtaining a second lifetime of the oscillatory event of the complex in the presence of the analyte;
(b3) comparing the first lifetime and the second lifetime to determine if the analyte extends the oscillatory event of the complex.

The present disclosure further provides a system for selecting a drug candidate for treating or preventing or delaying the onset or progression of an amyloidogenic disease, comprising: Aβ42;
an amphiphilic liposaccharide or a part thereof, wherein the amphiphilic liposaccharide or the part thereof is capable of forming a complex with Aβ42; and an apparatus for observing an oscillatory event of the complex.

In some embodiments, the amphiphilic liposaccharide comprises a lipid A part and an O-antigen part. In some embodiments, the amphiphilic liposaccharide is selected from the group consisting of LPS, MPL, PIX, PXI and salts thereof.

In some embodiments, the amphiphilic liposaccharide or the part thereof is anchored on a sensor of the apparatus. In some embodiments, the sensor is a solid sensor. An example of the solid sensor includes, but is not limited to, a gold sensor. In some embodiments, the amphiphilic liposaccharide or the part thereof is anchored on the sensor of the apparatus through a linker. An examples of the linker includes, but is not limited to, 3-mercaptopropanyln-hydroxysuccinimide ester.

In some embodiments, the apparatus comprises a mass-sensitive sensor. In some embodiments, the apparatus comprises a quartz crystal microbalance. In some embodiments, the apparatus acquires frequency signals and converts the frequency signals to mass signals.

The present disclosure also provides a method for clearance of Aβ42 in a subject in need, comprising administering a therapeutically effective amount of morin or a pharmaceutical composition comprising the same to the subject.

The present disclosure further provides a method for treating or preventing or delaying the onset or progression of an amyloidogenic disease in a subject in need, comprising administering a therapeutically effective amount of morin and an amphiphilic liposaccharide or a part thereof or a pharmaceutical composition comprising the same to the subject, wherein the morin and the amphiphilic liposaccharide or a part thereof are co-administered simultaneously, separately or sequentially or co-administered in combination as a coformulation, and the amphiphilic liposaccharide or the part thereof is capable of forming a complex with Aβ42.

In some embodiments, the amyloidogenic disease is selected from the group consisting of Alzheimer's disease, mild cognitive impairment, Parkinson's disease with dementia, Down's syndrome, diffuse Lewy body disease, cerebral amyloid angiopathy, vascular dementia, and mixed dementia.

### BRIEF DESCRIPTION OF DRAWINGS

FIG. 1 shows that ¹H NMR (400 MHz) spectra were measured in D₂O to monitor the substitution reaction between NHS-linker and LPS. The single peak at 1.92 ppm that originated from the four protons of the leaving group (NHS) shifted to 2.80 ppm, which might illustrate the amide bond formation between the linker (5 mg) and LPS (5 mg) in D₂O (450 μL) overnight.

FIG. 2 shows that the first bait (LPS) anchored on the gold sensor could be confirmed by trapping colistin (a cyclic peptide) via a host-guest interaction. Colistin is a cyclic peptide known to target LPS specifically. The mass augments in stage 2 indicated that the gold surface was indeed bound to LPS, leading to colistin adsorption. The upper image presents the order of the sample influx. Conditions: The flow rates of the two stages are 0.15 mL/min at 25° C. The concentrations of the linker and colistin are 0.1 mM and 20 μM, respectively. The solution at the final stage was changed to water for removing non-specific binding colistin.

FIG. 3 shows that an indirect method was used for finding the oscillation of the non-equilibrium interaction. Oscillation of the hydrophobicity of Aβ42 was noted by repeatedly adding freshly prepared LPS or aged LPS. The y axis of the graph represents the relative fluorescence intensity of the bis-ANS emission wavelength at 525 nm. Bis-ANS (4, 4′-Dianilino-1,1′-binaphthyl-5,5′-disulfonic acid) is sensitive to Aβ42, but not to LPS.

FIG. 4 shows that the linker (3-MCL-NHS) without conjugating LPS does not interact with Aβ42. The black curve shows original data from the frequency change once the gold sensor sensed the molecules. After the successful anchoring of the linker on the sensor surface, the resonant frequency could respond to time, showing an obvious downward trend. After calculation, the mass deposition of molecules on the gold surface increased instead as a time function. Two curves from the frequency and mass variation present a mirror-like image. Thus, for simplification, we omitted the change in the frequency variance and only showed the data of mass variation in the context figures. A washing step with LPS-free DI water was performed after finishing the linker adsorption on the gold sensor. Conditions: The flow rates in all stages are 0.15 mL/min at 25° C. The concentrations of the linker and Aβ42 are 0.1 mM and 20 μM, respectively.

FIGS. 5A and 5B show time evolution of the RMSD for LPS/Aβ42 pentamer mixture systems and two fibrillar structures, including 50QV (FIG. 5A) and 2NAO (FIG. 5B). Each conformation was simulated with four different initial configurations. The orange dashed line denotes the equilibration time for each system.

FIGS. 6A and 6B show fluorescence spectra of the dissolved tyrosine at the temperatures of 25-55° C. upon 275 nm excitation (FIG. 6A) and corresponding relative integrated fluorescence intensity difference at 300-445 nm upon 275 nm excitation (FIG. 6B). 125 and ΔI denote the fluorescence intensities at 25° C. and the difference at a temperature relative to that at 25° C., respectively. The dots and solid lines denote the observations and the linear progression fitting of the ratios of ΔI/I₂₅ with respect to ΔT. The concentration of tyrosine is 113 μM in 10 mM phosphate buffer at a pH of 7.4.

FIGS. 7A to 7E show that the apparent lifetime of the non-equilibrium interaction was determined by the duration required for mass dropping from 80% of its original quantity to 20% in FIG. 11A. The lifetime of each sample was indicated in colored lines. The corresponding values were listed in Table S1.

FIGS. 8A and 8B show that a concept based on editing the non-equilibrium oscillation acts as a drug-predicting ruler for the clearance of Aβ42 by neuronal cells. FIG. 8A: This putative diagram of the energy landscape represents the non-equilibrium state that might appear in the self-assembled proceeding pathway of Aβ42 by the oscillator (LPS) stimulation. FIG. 8B: The clearance time of neuronal cells may be increased from T₁ to T₁ plus T₂ by prolonging the non-equilibrium oscillation, resulting in the effective clearance of Aβ42.

FIGS. 9A to 9D show real-time observation of the autonomous non-equilibrium oscillation. FIG. 9A: Schematic of the design of a real-time detection platform for observing the non-equilibrium behavior of the autonomous LPS-Aβ42 interaction. The mass-sensitive sensor used lateral flow motion for sensing operation by two models-CFM and FSM. In both models, LPS was chemically anchored as an oscillator on the sensor's surface in the first stage. FIG. 9B: Continuous Aβ42 supplementation by CFM-based real-time measurement confirmed that the proceeding self-propagation of Aβ42 acts as a driving force to control the mutual interaction with LPS. FIG. 9C: The FSM-based real-time measurement revealed a non-equilibrium oscillation for the Aβ42-LPS interaction. FIG. 9D: An iteration of FSM-based real-time measurement confirmed the persistently oscillatory non-equilibrium behavior of the Aβ42-LPS interaction.

FIGS. 10A to 10C show programming of non-equilibrium oscillation for a biological emergency. FIG. 10A: The binding event of the heteromolecular interaction between LPS and the proceeding aggregates of soluble Aβ42 was tunable through the bait-specific strategy. The concentration of Aβ42 and three baits was 100 μM and 20 μM, respectively, for the real-time measurement. The inset showed a magnification of the rectangle's range. FIG. 10B: The rescue effect of LPS on the Aβ42-induced apoptosis of SH—SY5Y neuronal cells (comparing columns 1 and 2) completely disappeared when LPS was removed (comparing columns 3 and 4) and recovered once LPS was re-administered (comparing columns 5 and 6). Without co-treatment with LPS, the rescue effect of three baits against Aβ42-induced neuronal toxicity was ineffective (columns 7-9). FIG. 10C: The rescue effect of Aβ42-induced cytotoxicity could be observed when using the combination of low-dose LPS and morin. The concentration of Aβ42 for cell treatment was 100 μM. The concentrations of morin and CS were 1, 10, 50, and 100 μM. To evaluate the extension and shortening of LPS-trigged non-equilibrium in the presence of morin or CS, those of Aβ42-induced apoptosis were compared with that of LPS-trigged non-equilibrium in the absence of other molecules (column 4), not versus Aβ42-alone (column 3). Data are expressed as the mean±standard error of the mean (SEM) (n=3-6). *p<0.05, **p<0.01, ***p<0.001 by unpaired, two-tailed Student's t-tests.

FIGS. 11A and 11B show that a bait-intervened strategy prolonged the duration of non-equilibrium oscillation. FIG. 11A: The lifetime of the binding event between LPS and Aβ42 was prolonged in a dose-dependent manner for morin. FIG. 21B: Relationship of ln(γ) with ln([morin]). The corresponding linear regression manifested an R2 of 0.965.

FIGS. 12A to 12C show that this putative model presented the clearance efficiency of Aβ42 by prolonging the non-equilibrium oscillation, and Western blot analysis confirmed the hypothesis. FIG. 12A: The original strategy based on LPS yielded significant clearance of Aβ42. FIG. 12B: The bait-intervened strategy with the LPS-morin combination prolonged the acting time for the clearance of Aβ42. FIG. 12C: The bait-intervened strategy with the combination of low-dose LPS with morin resulted in dose-dependent depletion of Aβ42, and that with the LPSCS or LPS-EG combination had no impact on the degradation of Aβ42. Data were expressed as mean±SEM (n=6). *p<0.05, **p<0.01, ***p<0.001 by unpaired, two-tailed Student's t-tests.

### DETAILED DESCRIPTION OF THE INVENTION

Unless defined otherwise, technical and scientific terms used herein have the same meaning as commonly understood by one of ordinary skills in the art to which this disclosure pertains.

The articles “a” and “an” are used herein to refer to one or to more than one (i.e., to at least one) of the grammatical object of the article. By way of example, “an element” means one element or more than one element.

As used herein, the term “and/or” is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but has not yet experienced or displayed clinical or subclinical symptoms of the state, disorder or condition; and/or (2) inhibiting the state, disorder or condition, i.e., arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease.

The term “preventing” or “prevention” is recognized in the art, and when used in relation to a condition, it includes administering, prior to onset of the condition, an agent to reduce the frequency or severity of or delay the onset of symptoms of a medical condition in a subject relative to a subject which does not receive the agent.

As used herein, “onset” means the occurrence in a subject of clinical symptoms associated or consistent with a diagnosis amyloidogenic disease.

As used herein, “delay” in the onset or progression of a phase consistent with amyloidogenic disease means an increase in time from a first time point to onset or worsening of a phase consistent with amyloidogenic disease, such as cognitive impairment of the Alzheimer type. For example, a delay in the onset of amyloidogenic disease means that the onset of amyloidogenic disease, as defined herein, in a subject at risk to develop amyloidogenic disease is delayed from happening at its natural time frame by at least six months, 1 year, 1½ years, 2, years, 2½ years, 3 years, 3½ years, 4 years, 4½ years, 5 years, 5½ years, 6 years, 6½ years, 7 years, 7½ years or 8 years or more, and preferably from 3 years to 8 years and more preferably for 5 years after a normal cognitive subject has been determined to be at high risk to develop amyloidogenic disease. By way of further example, a delay in the progression of cognitive impairment that may progress to amyloidogenic disease or a delay in the progression of dementia means that the rate of cognitive decline is slowed relative to its natural time frame. These determinations are performed by using appropriate statistical analysis.

The term “amyloidogenic disease” includes any disease associated with (or caused by) the formation or deposition of insoluble amyloid fibrils. Exemplary amyloidogenic diseases include, but are not limited to AD, mild cognitive impairment, Parkinson's Disease with dementia, Down's Syndrome, Diffuse Lewy Body (DLB) disease, Cerebral Amyloid Angiopathy (CAA), vascular dementia and mixed dementia (vascular dementia and AD), amyloidosis associated with multiple myeloma, primary systemic amyloidosis (PSA), and secondary systemic amyloidosis with evidence of coexisting previous chronic inflammatory or infectious conditions. Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. PSA involves the deposition of insoluble monoclonal immunoglobulin (Ig) light (L) chains or L-chain fragments in various tissues, including smooth and striated muscles, connective tissues, blood vessel walls, and peripheral nerves.

In some embodiments, the disclosure especially provides a real-time detection platform to investigate the underlying mechanism of Aβ42 association and establish the dynamic oscillation as a drug-screening predictor for evaluating the clearance efficiency of neuronal cells. With the comprehensive study of the underlying molecular mechanisms, bridging the chemically dynamic behavior of Aβ42 as a privileged and beneficial characterization for biological essence might serve as a therapeutic strategy for drug development for amyloidogenic diseases.

Accordingly, the present disclosure provides a novel strategy for treating and/or preventing an amyloidogenic disease. In some embodiments, the present disclosure provides a method for selecting a drug candidate for treating or preventing or delaying the onset or progression of an amyloidogenic disease, comprising:
(a) contacting an analyte with Aβ42 and an amphiphilic liposaccharide or a part thereof, wherein the amphiphilic liposaccharide or the part thereof is capable of forming a complex with Aβ42; and
(b) determining if the analyte extends an oscillatory event of the complex; and the analyte is selected as the drug candidate.

The term “drug candidate” refers to any agent or compound that is suspected to have the ability to treat or prevent or delay the onset or progression of an amyloidogenic disease.

Without being limited by theory, it is believed that the dynamic oscillation implicated in structural heterogeneity during the self-assembly of Aβ42 may play a crucial role in eliciting cellular responses. In some embodiments of the disclosure, a real-time monitoring platform to observe an oscillatory non-equilibrium interaction that dominated the Aβ42 clearance by neuronal cells during interplay with an oscillator, such as the amphiphilic liposaccharide or the part thereof, is provided. For example, the electrostatic and hydrophobic segments of (LPS) are involved in the temporary heteromolecular association and slightly decelerated the intrinsic thermally-induced protein dynamics of Aβ42. A bait-specific intervention strategy could temporarily slow down the self-propagation of Aβ42 to extend the lifetime of autonomous oscillation and augment Aβ42 clearance of neuronal cells. The lifetime increment of oscillation shows a bait concentration-dependent manner to reflect the non-equilibrium binding strength. This relationship serves as a predictor for amyloidogenic disease drug discovery.

Without being limited by theory, it is believed that perturbation of the thermodynamic equilibrium, which causes dynamic non-equilibrium (oscillation), is a central dogma in programming of molecular self-assembly for creating new materials and maintaining life complexification. The involvement and appearance of oscillatory non-equilibrium behaviors in the use of molecule-scale building blocks for synthetic supramolecular systems are established. However, observing dynamic oscillation regarding one supramolecule-scale self-assembly paradigm with various intermediates and multiple reversible steps is rarely realized. For example, Aβ42, an aggregation-prone toxic polypeptide, contributes to senile plaques in the brain of individuals with AD. Before reaching a stable energy landscape, the self-propagation of Aβ42 adopts multi-step disassembly and reassembly that may rely on an interplay with other molecules in the blood for long scale self-elongation and striking a balance of entropy.

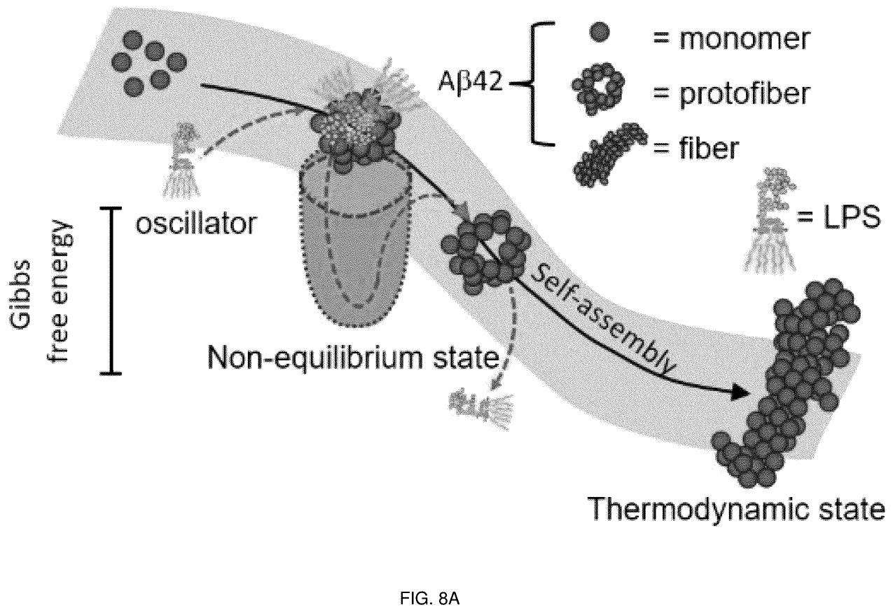

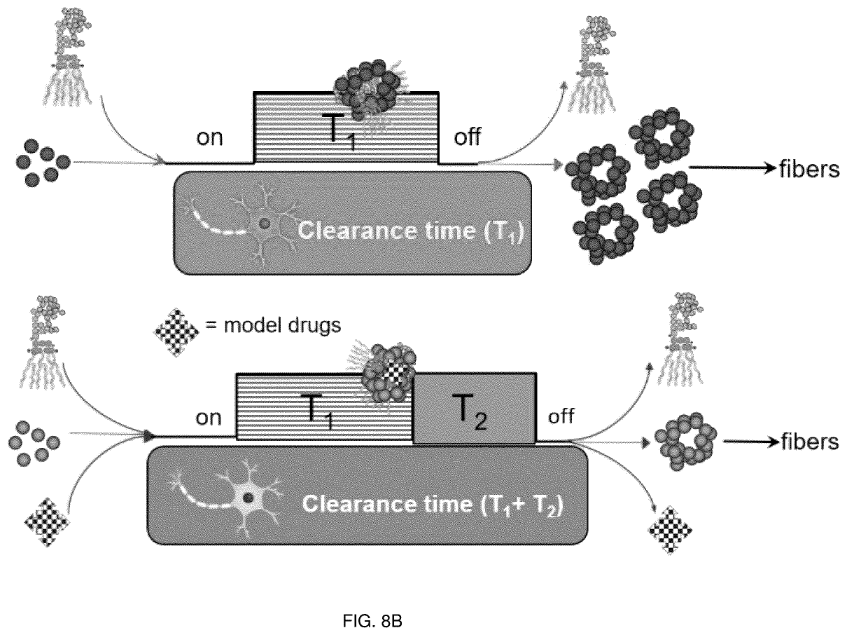

Notably, Aβ plaques examined in post-mortem brain sections from patients with AD were discovered to contain lipopolysaccharide (LPS), another aggregation-prone supramolecule derived from gram-negative bacteria. Although LPS is commonly associated with widespread inflammation in the brain, co-localization of LPS may be attributable to the antimicrobial activity of Aβ42 during the process of antimicrobial defense. A 30-min period heteromolecular association from the self-assembly of Aβ42 in the presence of LPS has been reported (T. H. Wu, R. H. Lai, C. N. Yao, J. L. Juang, S. Y. Lin, Angew. Chem. Int. Ed. 2021, 60, 4014-4017; Angew. Chem. 2021, 133, 4060-4063.). Such a temporary heteromolecular interaction may stimulate a robust cellular response in which toxic Aβ42 is cleared to prevent neurotoxicity. Accordingly, the amphiphilic liposaccharide or the part thereof such as LPS may play a key role in perturbing the thermodynamic self-assembly of Aβ42 to cause temporary non-equilibrium oscillation (FIG. 8A), consequently stimulating the clearance of Aβ42 by neuronal cells.

Owing to neuronal endocytosis occurring within less than 0.1 s, strategies for prolonging the lifetime of non-equilibrium oscillation may give neuronal cells more action time (clearance time) to increase the clearance of Aβ42, altering the extracellular homeostasis and reducing the accumulation of Aβ42. Considering the high failure rate and high cost of contemporary drug development for AD, the programming non-equilibrium oscillation capable of acting as an indicator for drug development to predict the clearance of Aβ42 by neuronal cells is essential. In some embodiments, the disclosure provides a bait-initiated intervention strategy to prolong the temporary interplay of two supramolecules (the amphiphilic liposaccharide or the part thereof and Aβ42) that might increase the clearance time (from T₁ to T₁ plus T₂) and consequently enhance the efficiency of Aβ42 clearance by neuronal cells (FIG. 8B). Molecular dynamics (MD) simulations and an intrinsic fluorescent transducer coupled with the temperature jump (T jump) system to decipher the atomic-level interaction between LPS and Aβ42 are also applied.

Examples of the analyte include, but are not limited to, a small molecule, a peptide, a polynucleotide, an antibody or biologically active portion thereof, a peptidomimetic, and a non-peptide oligomer.

As used herein, the term “amphiphilicity” refers to the property of one substance having both a hydrophobic site and a hydrophilic site. For example, when the medium is water, a substance having amphiphilicity forms micelle particles and the particles can be observed.

The liposaccharide disclosed herein refers to a compound comprising lipid, saccharide and lipid-saccharide conjugates. The liposaccharide may be derived from a natural source or may be artificial. An exemplary embodiment of the lipid is lipid A. Commonly, lipid A comprises two glucosamine (carbohydrate/sugar) units, in an β(1→6) linkage, with attached acyl chains (“fatty acids”), and normally contains one phosphate group on each carbohydrate. The lipid A as described herein may be modified. The term “oligosaccharide” refers to a carbohydrate structure having from 2 to about 7 saccharide units. The particular saccharide units employed are not critical and include, by way of example, all natural and synthetic derivatives of glucose, galactose, N-acetylglucosamine, N-acetylgalactosamine, fucose, sialic acid, 3-deoxy-D,L-octulosonic acid, and the like.

Particularly, examples of the amphiphilic liposaccharide include, but are not limited to, lipopolysaccharide (LPS), monophosphoryl lipid A (MPL), lipid A derivative IX (2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose) (denoted as PIX), or lipid A derivative XI (2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose) (denoted as PXI) or salts thereof.

The term “lipopolysaccharide” (LPS) refers to large molecules consisting of a lipid and a polysaccharide (glycophospholipid) joined by a covalent bond. LPS comprises three parts: 1) O antigen; 2) Core oligosaccharide, and 3) Lipid A. The O-antigen is a repetitive glycan polymer attached to the core oligosaccharide, and comprises the outermost domain of the LPS molecule. Core oligosaccharide attaches directly to lipid A and commonly contains sugars such as heptose and 3-deoxy-D-mannooctulosonic acid (also known as KDO, keto-deoxyoctulosonate). Lipid A is a phosphorylated glucosamine disaccharide linked to multiple fatty acids.

According to the disclosure, “monophosphoryl lipid A” is a detoxified endotoxin lipid A fraction, which lacks a saccharide and a phosphate group.

According to the disclosure, “lipid A derivatives IX and XI (denoted as PIX and PXI)” is a detoxified endotoxin lipid A fraction that belongs to one of MPL analogues that lacks two lipid chains.

The term “salts” includes any anionic and cationic complex, such as the complex formed between a cationic lipid disclosed herein and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfate, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

In some embodiments, the amphiphilic liposaccharide or the part thereof may induce a dissipative non-equilibrium state of self-aggregation for Aβ42. The amphiphilic liposaccharide or the part thereof forms a transient complex with Aβ42 through a non-equilibrium co-assembly process. The term “oscillatory event of the complex” may refer to an oscillating pattern of association and dissociation between the two molecules of the complex.

In some embodiments, the amphiphilic liposaccharide or the part thereof is capable of forming the complex with Aβ42, such as with Aβ42 monomers, protofibrils or oligomers, or fibers. Without being limited by theory, it is believed that the amphiphilic groove of Aβ42 protofibrils is important for complexing with the amphiphilic liposaccharide or the part thereof through a pattern of non-equilibrium behavior. In some embodiments, the amphiphilic liposaccharide or the part thereof may only form the complex with Aβ42 protofibrils.

In some embodiments, a 30-min period heteromolecular association from the self-assembly of Aβ42 in the presence of LPS has been observed. Such a temporary heteromolecular interaction may stimulate a robust cellular response in which toxic Aβ42 is cleared to prevent neurotoxicity. Hence, a drug candidate which is capable of extending the oscillatory event of the complex may give neuronal cells more action time (clearance time) to increase the clearance of Aβ42, altering the extracellular homeostasis and reducing the accumulation of Aβ42.

In some embodiments, the drug candidate is for clearance of Aβ42. That is, the drug candidate may achieve the purpose of treating or preventing or delaying the onset or progression of an amyloidogenic disease through clearance of Aβ42.

In some embodiments, step (a) comprises contacting the analyte with Aβ42 and the lipid A part or the O-antigen part of the amphiphilic liposaccharide.

In some embodiments, step (b) comprises observing the oscillatory events of the complex in the absence and presence of the analyte. In some embodiments, step (b) comprises:
(b1) obtaining a first lifetime of the oscillatory event of the complex in the absence of the analyte;
(b2) obtaining a second lifetime of the oscillatory event of the complex in the presence of the analyte;
(b3) comparing the first lifetime and the second lifetime to determine if the analyte extends the oscillatory event of the complex.

In some embodiments, step (b) comprises analyzing variation of mass signals over time to observe the oscillatory event of the complex. In some embodiments, step (b) further comprises acquiring frequency signals and converting the frequency signals to the mass signals.

In some embodiments, the amphiphilic liposaccharide or the part thereof serves as a bait. In some embodiments, the amphiphilic liposaccharide or the part thereof is anchored on a sensor, such as a mass-sensitive sensor. Aβ42 may be supplemented continuously to flow through the sensor in the absence of the analyte. The sensor may detect an increased mass signal when Aβ42 protofibrils bind to and associate with the bait, and may detect a decreased mass signal when Aβ42 protofibrils dissociate from the bait. Accordingly, the oscillatory event of the complex (the association and dissociation of the amphiphilic liposaccharide or the part and Aβ42) may be detected by the sensor as the variation of mass signals. In some embodiments, the mass-sensitive sensor may use a quartz crystal microbalance which can easily acquire frequency signals that could be directly converted to mass signals. The analyte may be supplemented with Aβ42 for observation of the oscillatory event of the complex in the presence of the analyte. The oscillatory events may be simplified into lifetimes for comparison. Details of observation of the oscillatory events and lifetime calculations are described in the Examples.

The present disclosure further provides a system for selecting a drug candidate for treating or preventing or delaying the onset or progression of an amyloidogenic disease, comprising: Aβ42;
an amphiphilic liposaccharide or a part thereof, wherein the amphiphilic liposaccharide or a part thereof is capable of forming a complex with Aβ42; and an apparatus for observing an oscillatory event of the complex.

In some embodiments, the amphiphilic liposaccharide or the part thereof is anchored on a sensor of the apparatus. In some embodiments, the sensor is a solid sensor. An example of the solid sensor includes, but is not limited to, a gold sensor. In some embodiments, the amphiphilic liposaccharide or the part thereof is anchored on the sensor of the apparatus through a linker.

In certain embodiments, the amphiphilic liposaccharide or the part thereof can be coupled to a linker to form a polysaccharide-linker intermediate in which the free terminus of the linker is an ester group. The linker is therefore one in which at least one terminus is an ester group. The other terminus is selected so that it can react with the amphiphilic liposaccharide or the part thereof to form the saccharide-linker intermediate. An example of the linker includes, but is not limited to, 3-mercaptopropanyln-hydroxysuccinimide ester.

The sensor may be a mass-sensitive sensor, such as a gold sensor. In some embodiments, the apparatus comprises a mass-sensitive sensor. For example, the apparatus may comprise a quartz crystal microbalance, which acquires frequency signals. In some embodiments, the apparatus converts the frequency signals to mass signals.

The present disclosure also provides a method for clearance of Aβ42 in a subject in need, comprising administering a therapeutically effective amount of morin or a pharmaceutical composition comprising the same to the subject.

The present disclosure further provides a method for treating or preventing or delaying the onset or progression of an amyloidogenic disease in a subject in need, comprising administering a therapeutically effective amount of morin and the amphiphilic liposaccharide or the part thereof or a pharmaceutical composition comprising the same to the subject, wherein the morin and the amphiphilic liposaccharide or the part thereof are co-administered simultaneously, separately or sequentially or co-administered in combination as a coformulation, and the amphiphilic liposaccharide or a part thereof is capable of forming a complex with Aβ42.

As used herein, the terms “patient,” “subject,” “individual,” and the like are used interchangeably, and refer to any animal, including any vertebrate or mammal, and, in particular, a human, and can also refer to, e.g., an individual or patient.

As used herein, the term “in need (of treatment)” refers to a judgment made by a caregiver (e.g., physician, nurse, nurse practitioner, or individual in the case of humans; veterinarian in the case of animals, including non-human mammals), and such judgment is that a subject requires or will benefit from treatment. This judgment is made based on a variety of factors that are in the realm of a caregiver's expertise, but that include the knowledge that the subject is ill, or will be ill, as the result of a condition that is treatable by the compounds of the present disclosure.

The term “administering” includes routes of administration which allow the agent of the disclosure to perform its intended function.

The term “therapeutically effective amount” of an agent as provided herein refers to a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

As used in the present disclosure, the term “pharmaceutical composition” refers to a mixture containing a therapeutic agent administered to an animal, for example a human, for treating or eliminating a particular disease or pathological condition that the animal suffers. In some embodiments of the disclosure, the pharmaceutical composition optionally comprises pharmaceutically acceptable excipients.

The term “pharmaceutically acceptable” as used herein refers to compounds, materials, compositions, and/or dosage forms which are, within the scope of sound medical judgment, suitable for use in contact with the tissues of a subject (either a human or non-human animal) without excessive toxicity, irritation, allergic response, or other problem or complication, commensurate with a reasonable benefit/risk ratio. Each carrier, excipient, etc., must also be “acceptable” in the sense of being compatible with the other ingredients of the formulation. Suitable carriers, excipients, etc., can be found in standard pharmaceutical texts.

The following examples are provided to aid those skilled in the art in practicing the present disclosure.

### EXAMPLES

A real-time mass-sensitive measurement strategy involving quartz crystal microbalance with dissipation explorer (QCM-D) and single-channel module (Q-Sense, Biolin Scientific, Vastra Frölunda, Sweden) was used to evaluate the non-equilibrium interaction between Aβ42 and two baits (LPS and natural compounds). In addition, the steady-state circular dichroism and fluorescence spectra upon excitation of tryptophan at different temperatures were recorded to reveal the secondary structures of Aβ42 in the presence and absence of LPS. The fluorescent T jump system was utilized to confirm the adsorbing capability of LPS to Aβ42 by reading the corresponding protein dynamics. The detailed methods and materials are described as follows.

### Platform for Monitoring the Non-Equilibrium Interaction

Chemicals and materials: The purities of amyloid peptides and all natural molecules, including chondroitin sulfate sodium (CS), epigallocatechin gallate (EG), morin (TCI), tannic acid, rosmarinic acid, quinacrine dihydrochloride, myricetin, 3-mercaptopropanyl-N-hydroxysuccinimide ester (MCL-NHS, MCE; a linker), and colistin, are higher than 99%, and therefore, further purification was not needed prior to use. Lipopolysaccharide (LPS; derived from Escherichia coli 0111:B4) and a 0.22 μm filter (non-pyogenic) were utilized. Fresh DI water was used to prevent LPS contamination from the environment for a non-specific interference in the mass-sensitive measurement.

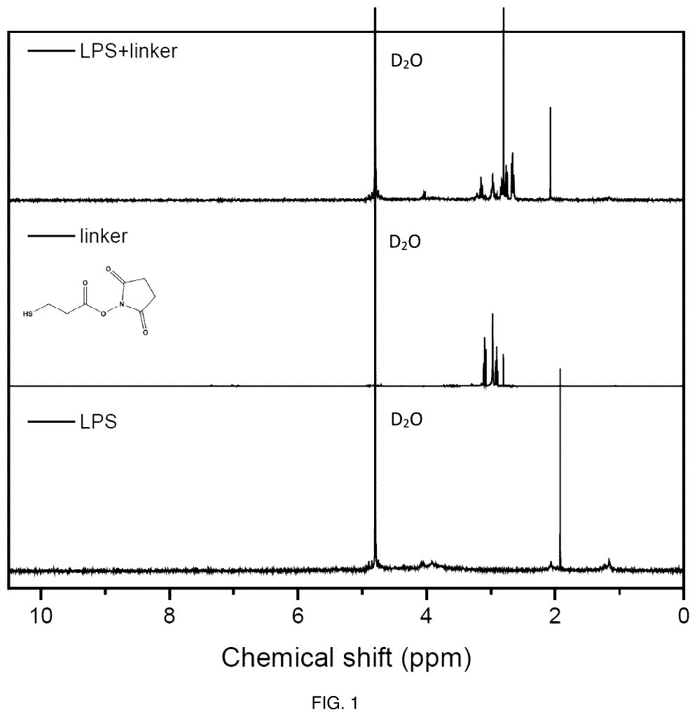

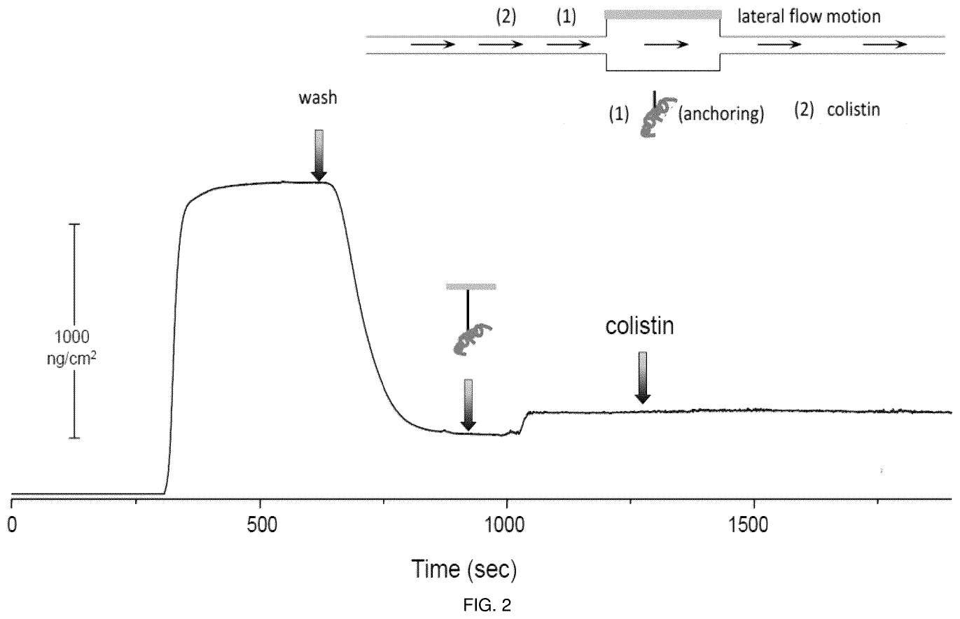

Preparation of the first bait: A solution of 0.1 mM MCL-NHS and LPS at a ratio of 1:1 was mixed in DI water. The mixture was cooled at 25° C. overnight (please refer to linker modification in the following), which spontaneously allowed the conjugation of the amine group of LPS and NHS-activated carboxylic acid of MCL. Owing to the structural complexity, it was difficult to validate the amide bond formation between MCL-NHS and LPS via NMR identification (FIG. 1). Nevertheless, the supramolecular host-guest recognition between LPS and colistin (a cyclic peptide) was used to identify the existence of MCL-modified LPS (first bait). The results are shown in FIG. 2. The details of the mass-sensitive measurement are described in the next section.

Linker modification: The conjugation of the first bait (LPS) and linker (3-mercaptopanyl-n-hydroxysuccinimide ester) was mixed together for overnight reaction before anchoring on the gold sensor. The linker may be linked to one or two —NH₂ groups in the core oligosaccharide part of LPS.

Real-time mass-sensitive measurement: Quartz crystal microbalance with dissipation explorer (QCM-D) and single-channel module (Q-Sense™, BIOLIN SCIENTIFIC®, Vastra Fralunda, Sweden) was used to evaluate the non-equilibrium interaction between Aβ42 and two baits (LPS and natural compounds). Prior to use, the new and reused gold sensors (a circle shape with a diameter of 1.0 cm, Q-Sense Sensors™, BIOLIN SCIENTIFIC®) were precleaned using a UV/ozone cleaner and piranha solution, which was a mixture of 30% H₂O₂ and concentrated H₂SO₄ at 1:3 (v/v). This piranha solution reacts violently with organic materials and should be handled with great care and protection. After washing of the gold sensors with LPS-free DI water, the first bait was initially inputted in a flow model at a flow rate of 0.15 mL/min under 25° C., which could be immediately anchored on a clean gold sensor. We conducted all measurements by switching between the continuous flowing model (CFM) and flowing-to-soaking model (FSM) after washing; the CFM maintained the same flow rate (0.15 mL/min), and the FSM allowed the flow velocity to slow down or completely stop. A sampling interval of 0.3 s was used to collect each frequency readout at seven overtones (1, 3, 5, 7, 9, 11, and 13 MHz), while the quartz crystal resonator of the QCN-D system settled at a fundamental resonant frequency (5 MHz). The Q-Sense system was utilized in addition to the following Sauerbrey equation to obtain the third overtone (n=3) and calculate the mass change after measurement.

$$Δ m = - \frac{Δ f \times C}{n} \qquad Eq . S1$$

where Δm is the change in the adsorbed mass of molecules; Δf is the time-responded frequency observed at the third overtone; and C is a constant that is equal to 17.7 ng/cm² at the resonance frequency of the quartz oscillator (5 MHz). The equation (Eq. S1) allows a direct quantitative study for mass comparison.

Operation of a real-time sensing platform: To perform the measurements, the LPS-free DI water and other solvents were allowed to continuously flow for about 1 h at 25° C. or until the baseline of two parameters (frequency and dissipation of QCM-D) reached an equilibrium state before sample operation and data collection. About 300 s after data collection, the solution containing the first bait was input into the system, which could anchor the thiol group of MCL-modified LPS on the gold surface of the sensors via Au—S bonding. Once the equilibrium saturation was reached, the LPS-free DI water was input into the system to remove any non-specific adsorption of the first bait. Subsequently, we used the CFM and FSM to study the non-equilibrium interaction between two baits (LPS and natural molecules). We confirmed a successful anchor of the MCL-modified LPS on the gold surface of the sensors (FIG. 2).

### In Vitro Test

Cell viability: Neuronal cells were maintained in MEM (GIBCO® 51200-038) supplemented with 10% FBS, 1×MEM NEAA (GIBCO® 11140-050), 1×L-glutamine (GIBCO® 25030-081), and 1×sodium pyruvate (GIBCO® 1136-070). Neuronal SH—SY5Y cells were plated at a density of 8,000 cells per well in 96-well plates and allowed to attach overnight at 37° C. in 5% CO₂ humidified air. Aβ42 (100 μM), LPS (10 nM), CS (100 μM), EG (100 PM), and morin (100 μM) were then added to each medium containing 2.5% dimethyl sulfoxide and sterilized using UV light. Thereafter, the solution containing Aβ42 and LPS was mixed with CS, EG, or morin at different concentrations ranging from 1 to 100 μM and added into the plate of the SH—SY5Y cells. The medium was then replaced, and the remaining mixture was incubated for 72 h at 37° C. in 5% CO₂ humidified air. Cell viability was measured using the CCK8 (SIGMA-ALDRICH® 96992) assay according to the manufacturer's protocol. Before the Aβ42 treatment of Neuronal SH—SY5Y cells, a 30 k filter was used to remove unbound LPS from the LPS-insulted Aβ42 mixture.

Western blot: The SH—SY5Y cells were plated at a density of 48,000 cells per well in 24-well plates and allowed to attach overnight at 37° C. in 5% CO₂ humidified air. Each group contained Aβ42 and either one bait or two baits and then incubated for 72 h at 37° C. in 5% CO₂ humidified air. To assess the level of Aβ42 in the cell culture medium, we collected the Aβ42 peptides from the entire volume of the cell culture medium and total cell lysates and mixed them with protein sample buffer (final 0.1 M Tris-HCl, pH 6.8, 10% glycerol, 2% SDS, 1% β-mercaptoethanol, and 0.01% bromophenol blue) for western blot analysis. The cell mixture samples were analyzed via 12% SDS-PAGE gel electrophoresis and transferred to a PVDF membrane. After blocking with buffer containing 5% w/v bovine serum albumin in PBST (1×phosphate-buffered saline and 0.1% Tween 20) at room temperature for 1 h, we incubated the membrane with a primary antibody diluted in the blocking buffer at 4° C. overnight. After hybridization with the primary antibody, the membrane was washed thrice with PBST before the addition of an HRP-conjugated secondary antibody against the primary antibody. The membrane was then washed thrice with PBST before immunoreactive bands were detected by UVP ChemStudio touch (ANALYTIK JENA®). The primary antibodies used in this study included the following: anti-Aβ (6E10, BIOLEGEND® 803001), anti-actin (GTX110564, GENETEX®), and anti-GAPDH (GTX100118). Signal intensity was analyzed using VisionWorks 9.1 and statistically analyzed using GraphPad Prism 9.

### Molecular Dynamic Simulations

To characterize the interaction between LPS and Aβ42 oligomer, we conducted molecular dynamic simulations of Aβ42 fibrillar pentamer with 10 LPS amphiphiles. Various Aβ42 fibril structures have been reported in the literature. Yet, most reported structures are fibrillar aggregates of Aβ42 fragments, and only a few of them correspond to full-length Aβ42 fibrils. Herein, we utilized two fibril structures of full-length Aβ42 with PDB ID of 50QV and 2NAO to generate two different Aβ42 fibrillar pentamer structures. The sequence of the O-antigen and the core of the LPS are illustrated in the linker modification section. The LPS structure was generated via CHARMM-GUI. To improve the sampling of LPS interacting with different sites of Aβ42 pentamers, we constructed four different initial configurations for each fibrillar conformation by randomly placing 10 LPS amphiphiles around the pentamer with an initial box size of 15×15×15 nm. Each box was further filled with water, and Na⁺ ions were then added to neutralize the system charges.

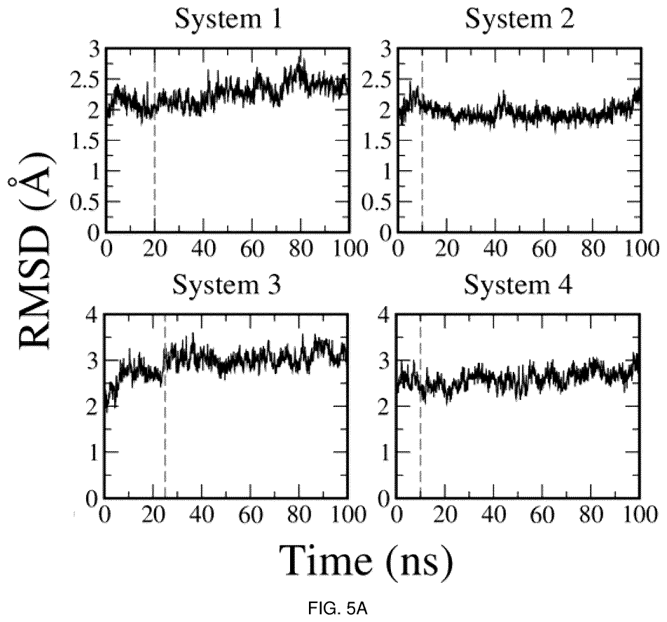

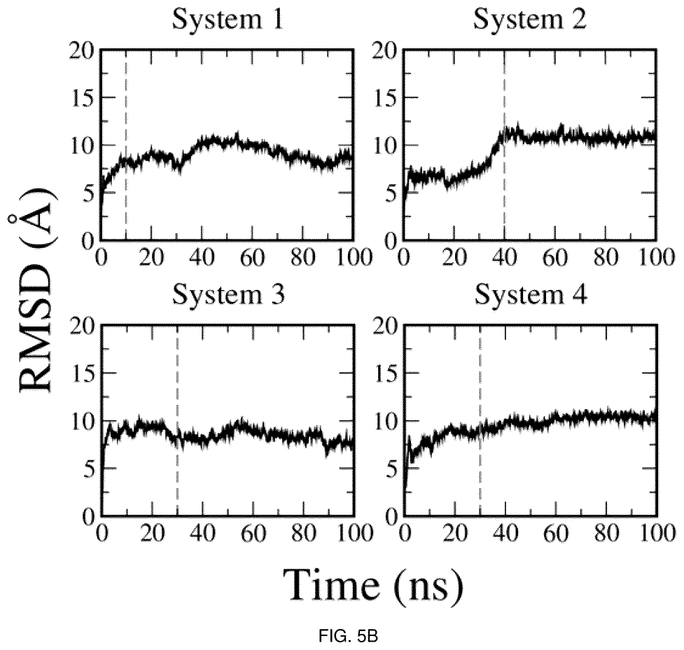

Atomistic molecular dynamic simulations were performed under the isothermal-isobaric (NPT) ensemble using the GROMACS 2016.4 package. Temperature and pressure were controlled at 310 K and 1 bar using the Nose-Hoover and Parrinello-Rahman algorithms, respectively. The CHARMM36 force field was applied for proteins, LPS, and ions. The TIP3P model was employed for the water molecules. Short-range interactions were cut off at 1.2 nm where Van der Waals interactions were smoothly shifted to 0 starting from 1.0 nm. The particle mesh Ewald method was used to evaluate long-range electrostatic interactions. The LINCS algorithm was used to constrain all bonds at their equilibrium lengths. A 2 fs time step was applied to integrate the equations of motions. Each simulation was equilibrated for 200 ns. System configurations were saved every 10 ps for analyses and visualization with the VMD package. Data analyses were performed when the root mean square deviation of the peptide backbone of the Aβ42 pentamer converged, as illustrated in FIG. 5.

The contact probability Pᵢ between LPS and the i^{th} residue of Aβ42 was defined as:

$${P}_{i} = \frac{1}{{N}_{sim}} {\sum }_{m}^{{N}_{sim}} \frac{1}{{N}_{m}} {\sum }_{n}^{{N}_{m}} \frac{1}{{N}_{p}} {\sum }_{k}^{{N}_{p}} δ ( {r}_{i} ) \qquad Eq . S 2$$

where δ(rᵢ) is a switch function representing the contact between LPS and the i^{th} residue when the minimal distance between them was less than 3 Å:

$$δ ( {r}_{i} ) = \{ \begin{matrix}1 , {r}_{i} \leq 3 Å \\ 0 , {r}_{i} > 3 Å\end{matrix} \qquad Eq . S3$$

The overall contact probability Pᵢ was thus averaged over Nₚ=5 peptides of the fibrillar pentamer, Nₘ frames of m^{th} simulation, and Nₛᵢₘ=5 replicas.

### Steady-State Spectroscopy

Ultraviolet-visible (UV-vis) absorption: The dissolved tyrosine and 300 μL stock Aβ42 solution were loaded in a quartz cuvette of an optical length of 2 mm (104002F-10-40, HELLMA ANALYTICS®). A spectrometer (USB4000-UV-Vis, OCEAN OPTICS®, 200-850 nm) with a deuterium lamp (SLS204, THORLABS®, 200-400 nm) was employed to record the spectra, with the 10 mM phosphate buffer solution as the background. The extinction coefficient of tyrosine at 276 nm of 1475 M⁻¹ cm⁻¹ and Aβ42 at 275 nm of 1410 M⁻¹ cm⁻¹ was used for quantifying the concentrations of the aforementioned samples with Beer's law.

Circular dichroism (CD): Approximately 300 μL of (96 μM) and mixtures of Aβ42 (96 μM) and LPS (160 nM) were loaded into a quartz cuvette of an optical length of 1 mm (110-1-40, HELLMA ANALYTICS®) and measured using a spectrometer equipped with a thermostat (Model 410, AVIV BIOMEDICAL®, Inc.) in 190-260 nm at 25° C. with a spectral resolution of 1 nm.

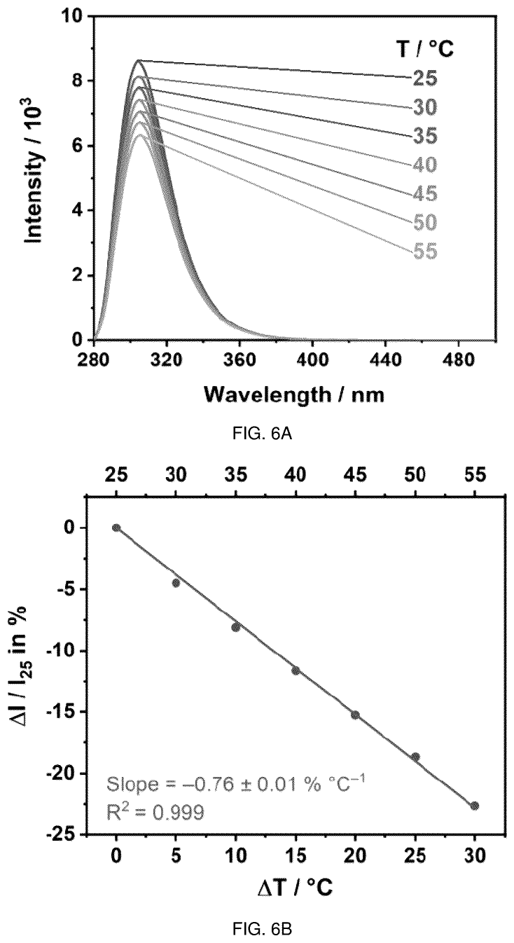

Fluorescence: The fluorescence spectra upon excitation at 275 nm were collected using a spectrometer (F-7000, HITACHI®) equipped with a thermostat (qpod & TC 125, QUANTUM NORTHWEST®) mounted at the sample compartment. A 2×10 mm² quartz cuvette (104002F-10-40, HELLMA ANALYTICS®) was used to load the 400 μL samples. The fluorescence spectra of Aβ42 (94 μM) and mixtures of Aβ42 (94 μM) and LPS (160 nM) were immediately collected at 25-45° C. after thermalization at the set temperatures. The normalized fluorescence spectra of Aβ42 (96 μM) and mixtures of Aβ42 (96 μM) and LPS (160 nM) were collected at 25° C. after 1 min thermalization at 25° C. The fluorescence spectra of the dissolved tyrosine (113 μM) at 25-55° C. were collected after 10 min thermalization at the set temperatures, as shown in FIG. 6A. All spectra were corrected by the fluorescence spectrum of 160 nM LPS solution in 10 mM 7.4 pH phosphate buffer to exclude the minor contribution of the Raman signal of H₂O at 305 nm.

### Fluorescent T Jump

A fluorescent T jump system was constructed on the basis of a previous report (Yang, C. T., Chu, L. K. Phys. Chem. Chem. Phys. 2022, 24, 11079-11085), with slight modification. The aqueous solutions were sandwiched by two quartz windows (diameter=25 mm, thickness=2 mm, #14-962, EDMUND OPTICS®) separated by a drilled Teflon spacer of a thickness of 0.8 mm. The diameter of the hole in the center was 2.0 mm. The circular infrared beam, which was used to heat up water to increase the temperature, from a continuous-wave diode laser at 1550 nm was focused with two convex lenses (f=100.0 mm; f=75.6 mm) to a diameter of 1.6 mm before entering the sample cell. A mechanical shutter was placed in between the lenses to control the exposure timing. A diverse UV light from a 275 nm LED lamp after passing a UV diffuser and an iris diaphragm was further focused with a lens pair (f=25.0 mm; f=40.0 mm) to the sample cell in the same direction of infrared beam propagation at an angle of ca. 450 to excite tyrosine and Aβ42. The fluorescence was collected using a MicroSpot Focusing Objective (LMU-20X-NUV, THORLABS®, 325-500 nm) and later focused through a 200 μm pinhole by a lens (f=50.0 mm) to an optical fiber (300-1,200 nm). Thereafter, the collected fluorescence light was introduced to a photomultiplier tube and filtered using an optical filter to define the detection range at 300-445 nm. The voltage signal from the photomultiplier was amplified by a factor of 20 using a voltage pre-amplifier, and further recorded using an oscilloscope.

### Derivation of the Temperature from the Evolution of Fluorescence Intensity Change of Tyrosine Upon T Jump

According to Gally and Edelman, the temperature increase reduced the fluorescence intensity of tyrosine. The fluorescence spectra of the dissolved tyrosine at different temperatures are shown in FIG. 6A. The relative fluorescence change in response to the temperature increment with respect to 25° C. (FIG. 6B) was −0.76% ° C.⁻¹. Thus, the fluorescence intensity change of tyrosine during infrared laser heating can be used as a fluorescent thermometer. To account for the fluorescence intensity drop due to photobleaching, we collected the fluorescence intensity evolutions of the dissolved tyrosine in the absence and presence of 1550 nm laser; their fluorescence intensity change evolutions,

$$\frac{Δ I ( t )}{{I}_{0}} ,$$

can be derived using Eq. S4:

$$\frac{Δ I ( t )}{{I}_{0}} = \frac{I ( t ) - {I}_{0}}{{I}_{0}} = \frac{V ( t ) - {V}_{0}}{{V}_{0}} \times 100 \% \qquad Eq . S4$$

I₀ and I(t) denote the mean fluorescence intensity before starting the T jump and the fluorescence intensity evolution after the T jump, respectively. They are proportional to the voltages from the photomultiplier, in terms of V₀ and V(t), respectively. The temperature change evolution, ΔT(t), can be derived using Eq. S5:

$$Δ T ( t ) = \frac{1}{α} Y ( t ) = \frac{1}{α} [ ( \frac{{V}_{Tyr} ( t ) - {V}_{0 , Tyr}}{{V}_{0 , Tyr}} ) IR laser on - ( \frac{{V}_{Tyr} ( t ) - {V}_{0 , Tyr}}{{V}_{0 , Tyr}} ) IR laser off ] \qquad Eq . S5$$

where Y(t) and α denote the corrected fluorescence intensity change evolution and conversion factor −0.76% ° C.⁻¹, respectively, for deriving ΔT(t).

### Evolution of Fluorescence Intensity Change of Aβ42 and LPS-Aβ42 Upon T Jump

The photobleaching-induced fluorescence decrease should be considered in the evolution of the fluorescence intensity change of Aβ42 and LPS-A342 upon T jump. Eqs. S6 and S7 were used to derive the corrected fluorescence intensity change evolution of Aβ42 and LPS-Aβ42, in the forms of A(t) and L(t), respectively:

$$A ( t ) = ( \frac{{V}_{A β 42} ( t ) - {V}_{0 , A β 42}}{{V}_{0 , A β 42}} ) IR laser on - ( \frac{{V}_{A β42} ( t ) - {V}_{0 , A β 42}}{{V}_{0 , A β 42}} ) IR laser off \qquad Eq . S6$$

$$L ( t ) = ( \frac{{V}_{A β42} ′ ( t ) - {V}_{0 , A β 42} ′}{{V}_{0 , A β 42} ′} ) IR laser on - ( \frac{{V}_{A β42} ′ ( t ) - {V}_{0 , A β 42} ′}{{V}_{0 , A β 42} ′} ) IR laser off \qquad Eq . S7$$

where V_{0,Aβ42} and V_{0,Aβ42}′ represent the mean voltage from the photomultiplier of Aβ42 and LPS-Aβ42 before starting the T jump; V_{0,Aβ42} (t) and V_{0,Aβ42}′(t) represent the voltage from the photomultiplier of Aβ42 and LPS-Aβ42 after the T jump. The A(t) and L(t) were obtained from the averages of three measurements.

### Kinetic Interpretations

A two-state model was proposed to extract the protein dynamics of Aβ42 upon T jump,

$$P \to Q \qquad Eq . S8$$

where P and Q denote the structure of Aβ42 before and after the temperature increase, respectively. Before the T jump, the concentration of P is [P]₀. After the T jump, the concentrations of P and Q at a given time, [P](t) and [Q](t), conform to the mass conservation law:

$$[ P ] ( t ) + [ Q ] ( t ) = {[ P ]}_{0} \qquad Eq . S9$$

Since the fluorescence intensity is proportional to the concentration of P and Q, the measured fluorescence intensity before and after the T jump, written as I_{0,Aβ42} and I_{0,Aβ42} (t), can be expressed using the following equations:

$${I}_{0 , A β 42} = {[ P ]}_{0} {ϕ}_{P} \qquad Eq . S10$$

$${I}_{A β 42} ( t ) = [ P ] ( t ) {ϕ}_{P} ( 1 + Δ T ( t ) {α}_{P} ) + [ Q ] ( t ) {ϕ}_{Q} ( 1 + Δ T ( t ) {α}_{Q} ) \qquad Eq . S11$$

where ϕr and ϕg denote the apparent quantum yields of states P and Q, and α_{P} and α_{Q} denote the intensity-to-temperature conversion factors of Aβ42. During the T jump, the temperature slightly increases. Thus, the term 1+ΔT(t)α_{P,Q} and α_{Q} was included to account for the fluorescence intensity change of Aβ42 in Eq. S11. Further, α_{P} and α_{Q} could be similar because the linear progression did not reveal a significant deviation. Changing the temperature (i.e., populations of P and Q) does not alter the intensity-to-temperature conversion factors α_{P} and α_{Q}. Thus, it is reasonable to assume that α_{P}=α_{Q}. Accordingly, Eq. S11 can be rewritten in the following form:

$${I}_{A β 42} ( t ) = ( 1 + Δ T ( t ) {α}_{P} ) ( [ P ] ( t ) {ϕ}_{P} + [ Q ] ( t ) {ϕ}_{Q} ) \qquad Eq . S12$$

After the correction of photobleaching using Eq. S6, the fluorescence intensity change evolution of Aβ42, A(t), is explicitly expressed using Eq. S13:

$$A ( t ) = \frac{{I}_{A β42} ( t ) - {I}_{0 , A β42}}{{I}_{0 , A β42}} = \frac{( 1 + Δ T ( t ) {α}_{P} ) ( [ P ] ( t ) {ϕ}_{P} + [ Q ] ( t ) {ϕ}_{Q} )}{{[ P ]}_{0} {ϕ}_{P}} - 1 \qquad Eq . S13$$

Thus, the thermally induced protein dynamics of Aβ42, D(t), can be expressed using Eq. S14:

$$D ( t ) = \frac{[ P ] ( t ) {ϕ}_{P} + [ Q ] ( t ) {ϕ}_{Q}}{{[ P ]}_{0} {ϕ}_{P}} = \frac{A ( t ) + 1}{1 + Δ T ( t ) {α}_{P}} \qquad Eq . S14$$

By replacing ΔT(t) using Eq. S5, D(t) of Aβ42 is rewritten as Eq. S15,

$$D ( t ) = \frac{A ( t ) + 1}{\frac{{a}_{P}}{a} Y ( t ) + 1} = \frac{A ( t ) + 1}{\frac{47}{76} Y ( t ) + 1} \qquad Eq . S15$$

where α_{P} and α_{Q} are −0.47% ° C.⁻¹ and −0.76% ° C.⁻¹ for Aβ42 and tyrosine, respectively. The D(t) of Aβ42 was therefore derived using Eq. S15. Upon similar treatment, the D(t) of LPS-Aβ42 was written as Eq. S16 and further used to derive the corresponding D(t).

$$D ( t ) = \frac{L ( t ) + 1}{\frac{49}{76} Y ( t ) + 1} \qquad Eq . S16$$

### Example 1

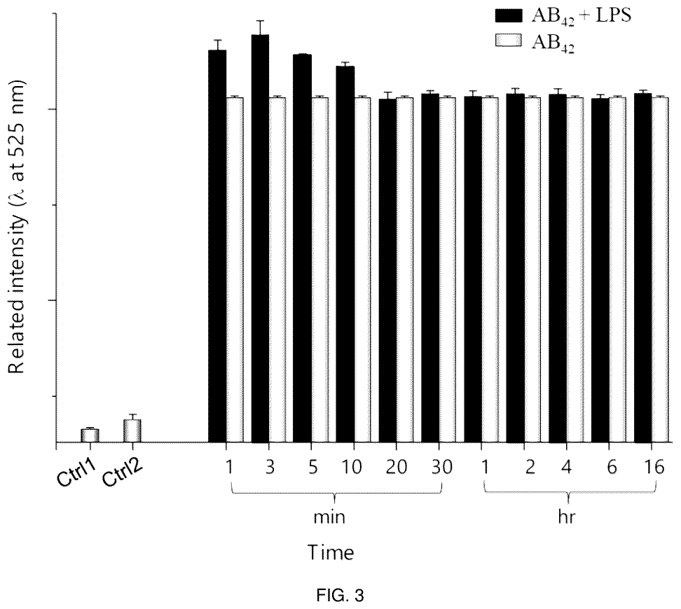

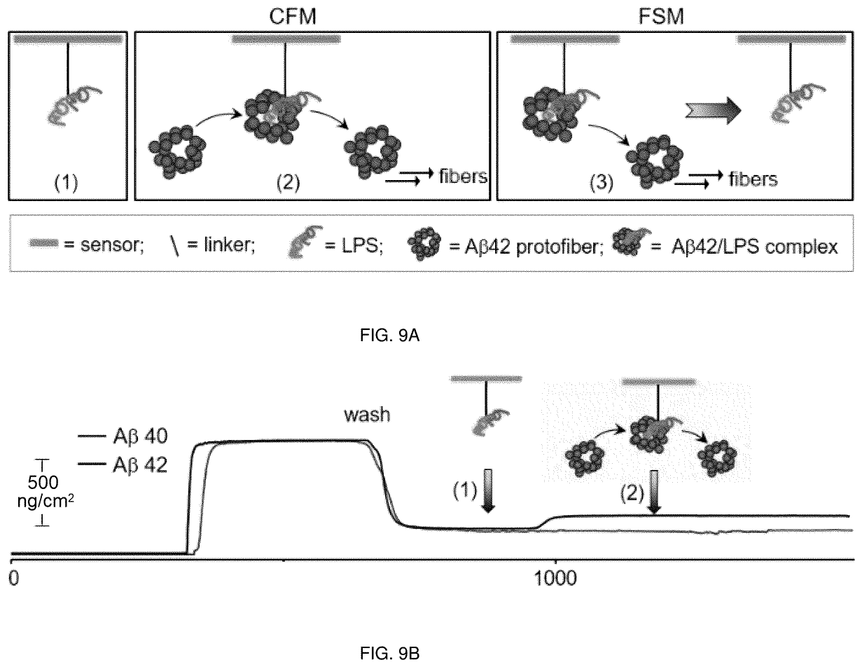

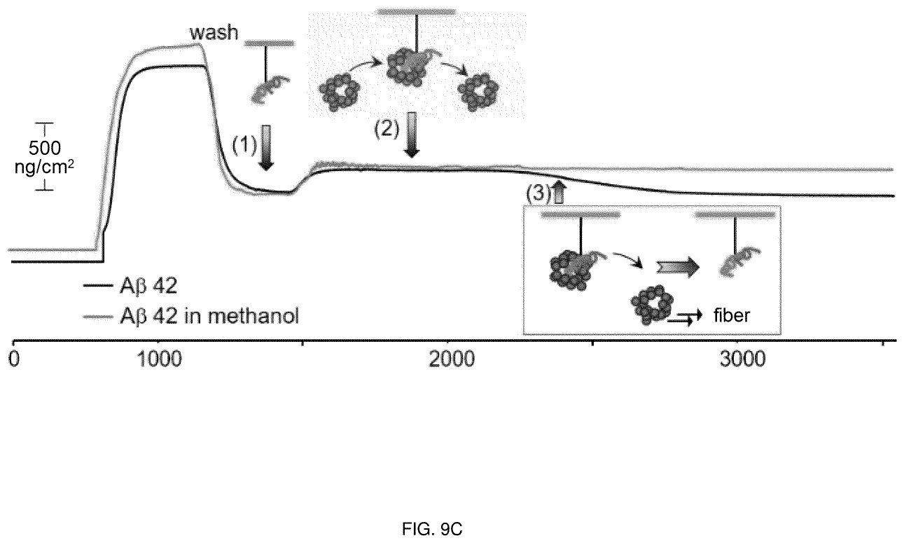

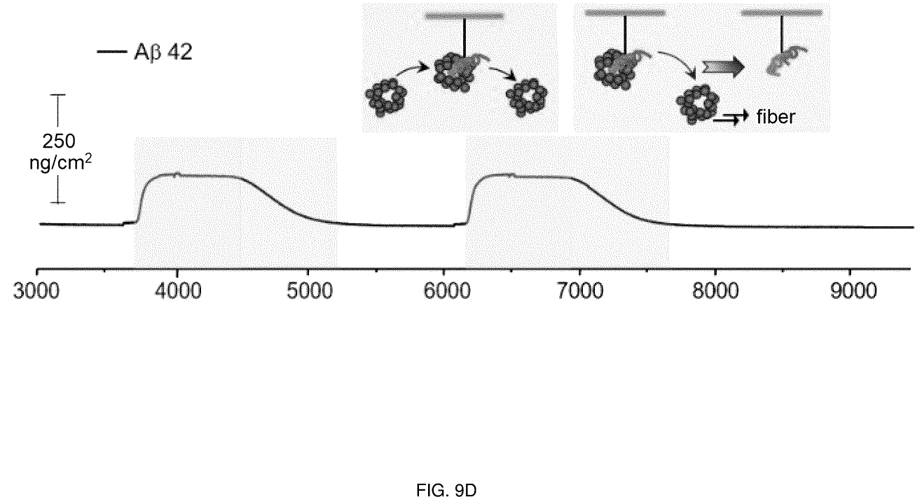

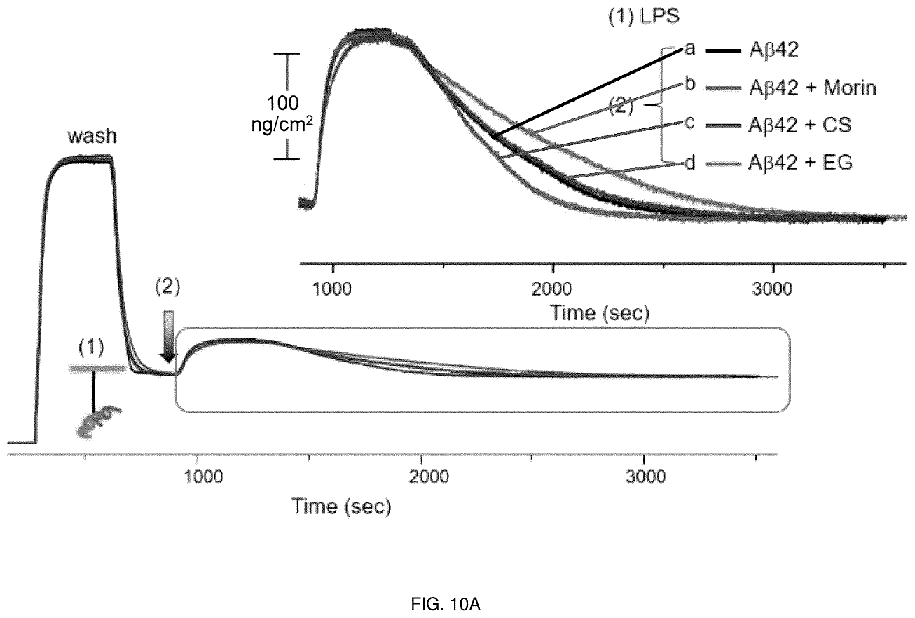

To observe and describe the oscillation behaviors between LPS and Aβ42, we developed a real-time analytical system comprising two models, a continuous flowing model (CFM) and a flowing-to-soaking model (FSM). The concept, which ensures a sufficient influx of Aβ42, is recapitulated in FIG. 9A. The FSM can pause the flowing of solution, which could allow the desired self-assembly of Aβ42 in the sensing containers, but without inputting fresh Aβ42. For validation, we utilized a thiol-containing linker to anchor a chemical oscillator (LPS) to the gold surface of a mass-sensitive sensor for capturing Aβ42 (FIG. 9A, frame 1). The Supporting Notes and FIGS. 1 and 2 present evidence for modifying the oscillator, and detail the surface-anchoring process of the analytical platform. In FIGS. 9B and 9C, arrow 1 illustrates an obvious increase in mass, indicating successful chemical fixation of LPS on the surface of the sensors. The first step entailed washing the sensor's surface to eliminate non-specific adsorption; the detailed protocol is depicted in the Supporting Notes. Subsequently, we applied the CFM (FIG. 9A, frame 2) or FSM (FIG. 9A, frame 3) to perform second-stage measurement while allowing the solution containing Aβ42 to flow persistently, and observed an elevated mass signal corresponding to the LPS-Aβ42 specific interaction (FIG. 9B/9C, arrow 2). The sensor could still detect the Aβ42-LPS interaction despite the continuous flowing in and out of Aβ42 caused by the CFM. While switching to the FSM, we observed a noticeable mass damping (FIG. 9C, “Aβ42” line, arrow 3), although the sensors remained soaked in the solution containing Aβ42. The up-and-down oscillation (FIG. 9C, “Aβ42” line, arrows 2 and 3) in the mass signal might have originated from the proceeding self-assembly of Aβ42 that became a driving force for the LPS-A342 disassociation. To further investigate this possibility, we dissolved Aβ42 in methanol, a solvent without hydrogen binding that might slow down the self-assembly, and discovered that the mass damping completely disappeared (FIG. 9C, “Aβ42 in methanol” line). Additionally, the lifetime of mass oscillation was less than 30 min, which was consistent with an indirect observation by a specific fluorescent probe (FIG. 3), and the non-equilibrium oscillation could be repeated after the flow of the Aβ42 solution had been re-initiated (FIG. 9D). This result illustrated that LPS was a specific oscillator to induce dynamic oscillation during the self-assembly of Aβ42.

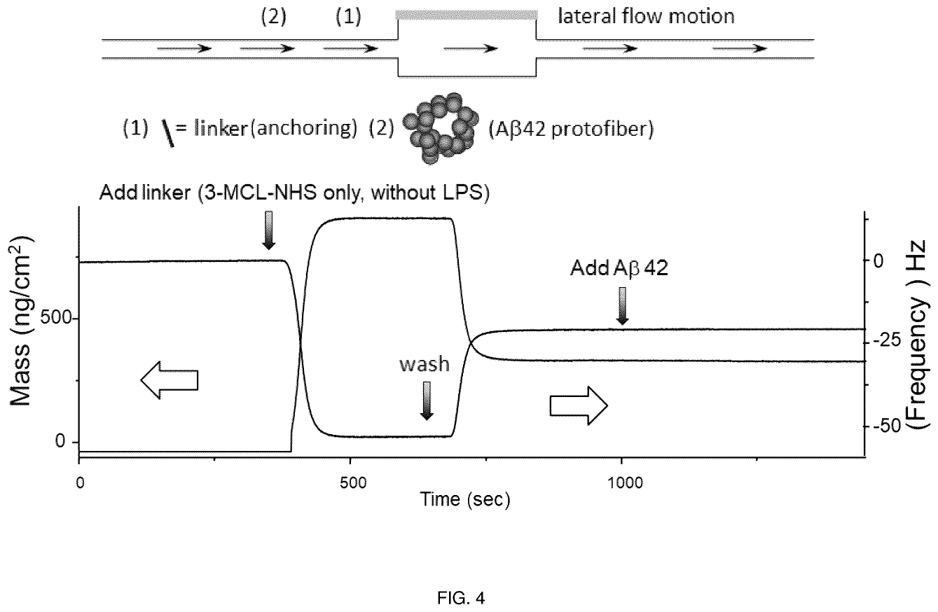

The energy landscape of supramolecular amyloid-level dynamics is complex, and only has an in-silico computational study that predicted numerous kinetically trapped states in the thermodynamic pathway of the proceeding self-propagation of Aβ42, with these states promoting long-scale elongation. However, a non-equilibrium state may have existed in the free energy landscape of the proceeding self-propagation pathways of Aβ42, as indicated by a dissipative configuration. Since the state showed that it was maintained by the continuous influx and efflux of substances (herein is LPS) from the surrounding stimulation, the behavior was attributable to non-equilibrium oscillation. We determined that the phenomenon was a time-limited event that was not readily amenable to isothermal titration calorimetry. The mass-sensitive sensor using a quartz crystal microbalance easily acquired frequency signals that could be directly converted to mass signals using the Sauerbrey equation [Equation S1], which may provide evidence of a heteromolecular association. The conversion from frequency data to mass signals is described in detail in the Supporting Notes. The example in FIG. 4 illustrates an inverted contour between frequency and mass plots.

We then performed MD simulations and T jump fluorescence measurements to further elucidate the Aβ42-LPS interaction. Previous simulation and theoretical studies have proposed various nucleus sizes of Aβ42 protofibrils: ranging from 3 to 50 monomers for fibrillization. We selected an Aβ42 pentamer with favorable structural stability as the fibrillar nucleus for the simulation study. Considering the polymorphic nature of Aβ42, we utilized two Aβ42 pentamer conformations from the protein data bank; they had PDB IDs of 50QV and 2NAO, respectively. To improve the sampling of LPS interacting with different sites of Aβ42, we conducted simulations of four initial configurations of 10 LPS molecules mixed randomly with the pentamer for each oligomer configuration.

The contacts between LPS and Aβ42 were analyzed from equilibrated MD trajectories for which the system equilibration was verified by the convergence of root mean squared deviation of Aβ42 pentamer (FIG. 5). Detailed simulation setups are discussed in the Supporting Notes. The simulation results indicated that LPS absorption occurs on the Aβ42 pentamer in either 50QV or 2NAO conformation. We found that LPS interacts with Aβ42 using its hydrophilic O-antigen and core oligosaccharide groups as well as its hydrophobic alkyl tails of lipid A; this is consistent with previous experimental observations. For the 50QV pentamer, analyses of the contact probability illustrate that LPS mainly interacts with the segment around K16 and the hydrophobic segment near the C-terminus of Aβ42. Specifically, negatively charged LPS can interact with K16 via electrostatic attraction. Such attraction further drives the close contacts between LPS alkyl tails and the nearby hydrophobic residues of L17 to A21. The alkyl tails of LPS can also interact with the hydrophobic segment of G38 to 141 at the Aβ42 C-terminus, driving the adsorption of LPS on the Aβ42 pentamer. For the 2NAO pentamer, LPS mainly interacts with Aβ42 at the hydrophobic segment of 132 to M35 near the C-terminus and the hydrophobic A2 and F4 at the N-terminus. Combining the results from both Aβ42 conformations indicated that electrostatic attraction and the hydrophobic effect are critical to the association between LPS and Aβ42. Conversely, Y10 is not involved in the electrostatic or hydrophobic interaction between LPS and Aβ42.

We also used the observed steady-state fluorescence and fluorescent T jump, which are capable of providing the dynamic information of the structural alteration upon a thermal stimulus, to demonstrate the proposed dynamic interaction capability. The circular dichroism (CD) spectra of Aβ42 and LPS-Aβ42 were highly similar and manifested the characteristics of 3-sheet in terms of biphasic ellipticity (θ) at 217 nm (negative θ) and <200 nm (positive θ), suggesting that the presence of LPS does not initiate the unfolding of the secondary structure of Aβ42. This result is consistent with the MD simulations that LPS merely attaches to Aβ42 rather than causing unfolding. Additionally, the normalized fluorescence spectra of Aβ42 and LPS-Aβ42 are pretty similar, but still coupled with a minute difference at 340-400 nm. The fluorescence spectra of Aβ42 and LPS-Aβ42 at 25-45° C. upon 275 nm excitation of the constituent tyrosine (Y10) show that the fluorescence intensity decreases as the temperature increased without significant spectral shifts. Additionally, their relative fluorescence changes in response to the temperature increment are 0.47 and 0.49%° C.⁻¹, respectively, and differ from the temperature response of the dissolved tyrosine (0.76%° C.⁻¹, a controlled experiment from tyrosine only) presented in FIGS. 6A and 6B. This result suggested that the chemical environments of the constituent tyrosine in Aβ42 and LPS-Aβ42 were similar, but quite different from that of dissolved tyrosine at aqua. However, the steady-state approach did not reveal the dynamic properties of Aβ42 in the presence of LPS.

Since the Y10 did not directly interact with LPS according to our MD simulations, the tyrosine fluorescent T jump was employed to probe the dynamic difference in the structural relaxation of Aβ42 and LPS-Aβ42, instead of the LPS-tyrosine interaction. Upon infrared heating water, the fluorescence intensity of the dissolved tyrosine decreased, reflecting an increasing trend in temperature. However, the fluorescence intensity change evolutions of Aβ42 and LPS-Aβ42 differ from that of the dissolved tyrosine at the later period of the curve profile, which mainly referred to the structural alteration at the vicinity of Y10 other than the temperature increase. In the analysis using the two-state model, the resultant protein dynamics of Aβ42 manifested a rapid and small response to temperature—a downward feature, followed by a slow upward response. However, the presence of LPS retarded the upward response. This finding might also suggest that the attachment of LPS decelerated the structural alteration of the Aβ42 aggregate during fibrillization.

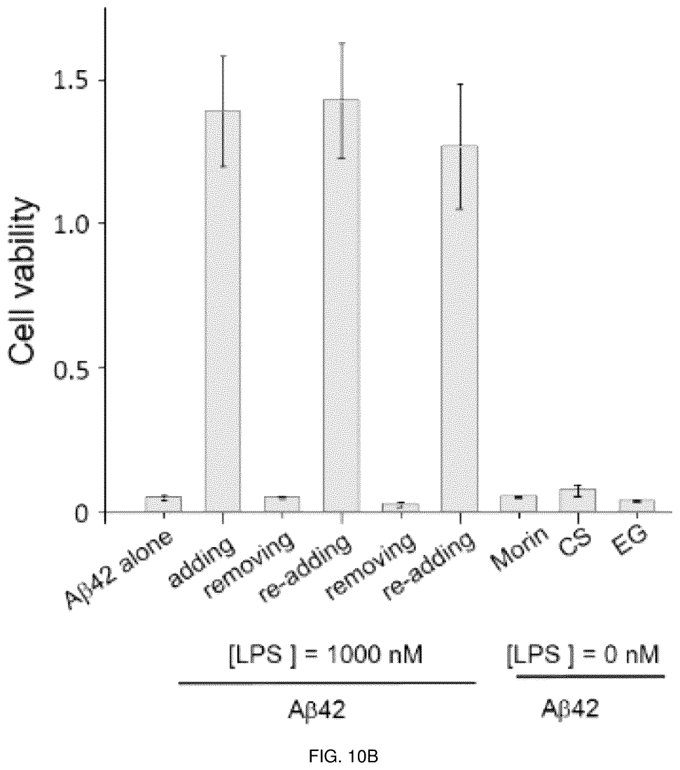

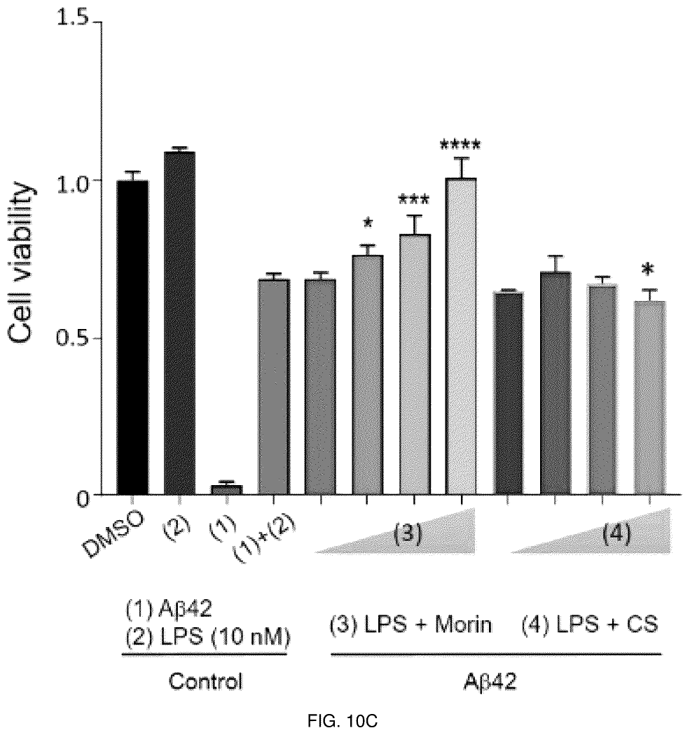

Considering the possible implications of a non-equilibrium emergency in nature, we monitored signal oscillation and its programmable potentials by either speeding up or slowing down the proceeding self-assembly of Aβ42. Some natural molecules of polyphenols and flavonoids-including morin, chondroitin sulfate (CS), epigallocatechin gallate (EG), rosmarinic acid, tannic acid, quinacrine dihydrochloride, curcumin and myricetin—have been reported to intervene in the aggregation of Aβ42. Thus, we used these small molecules (bait) in combination with a low dose of LPS (oscillator) to form a bait-intervened strategy to screen for the possibility of programming the non-equilibrium oscillation of Aβ42. Notably, the oscillatory event of the LPS-Aβ42 complex could only be extended in the combination of LPS plus morin (FIG. 10A, lines a vs. b). On the contrary, the combination of LPS plus CS shortened the binding event (FIG. 10A, lines a vs. c). The combination of LPS plus other molecules, including EG (FIG. 10A, line d), rosmarinic acid, tannic acid, quinacrine dihydrochloride, curcumin and myricetin, did not perturb the oscillatory event of the LPS-Aβ42 complex.

Subsequently, we evaluated whether the dynamic oscillation of Aβ42 by using the bait-intervened strategy to prolong or shorten the oscillation could influence the rescue outcomes of neuronal cells. First, it is essential to clarify whether LPS is capable of destroying toxic oligomers of Aβ42. After adding a high dose (1000 nM) of LPS to SH—SY5Y neuronal cells, the viability of neuronal cells in medium containing Aβ42 reverted to 100% (FIG. 10B, column 2 vs. column 1). Conversely, the cell viability decreased to less than 10% when the LPS of Aβ42 solution was removed (FIG. 10B, columns 2-3). If the LPS-treated Aβ42 could destroy toxic oligomers, we would not have observed the reverse of Aβ42-induced apoptosis (FIG. 10B, columns 1-6). The finding indicated that the transient interaction was not capable of destroying the toxic oligomers of Aβ42. We determined that that small baits alone—including morin, CS, and EG—could not alleviate Aβ42-induced cell death (FIG. 10B, columns 7-9). Interestingly, when the medium contained a low dose (10 nM) of LPS, the rescue effect of Aβ42-induced cell apoptosis increases (FIG. 10C, columns 3-4) in a dose-dependent manner in the co-treatment of morin (FIG. 10C, columns 5-8 vs. column 4). However, this improvement in the rescue effect was not observed from the LPS—CS combination (FIG. 10C, columns 9-12). Accordingly, for Aβ42 non-equilibrium oscillation, the LPS-morin and LPS—CS combination led to significant prolongation and shortening (FIG. 10A, lines b vs. d), respectively. Considering these findings, we further investigated modifiable non-equilibrium oscillation that can be effectively applied in biological emergencies.

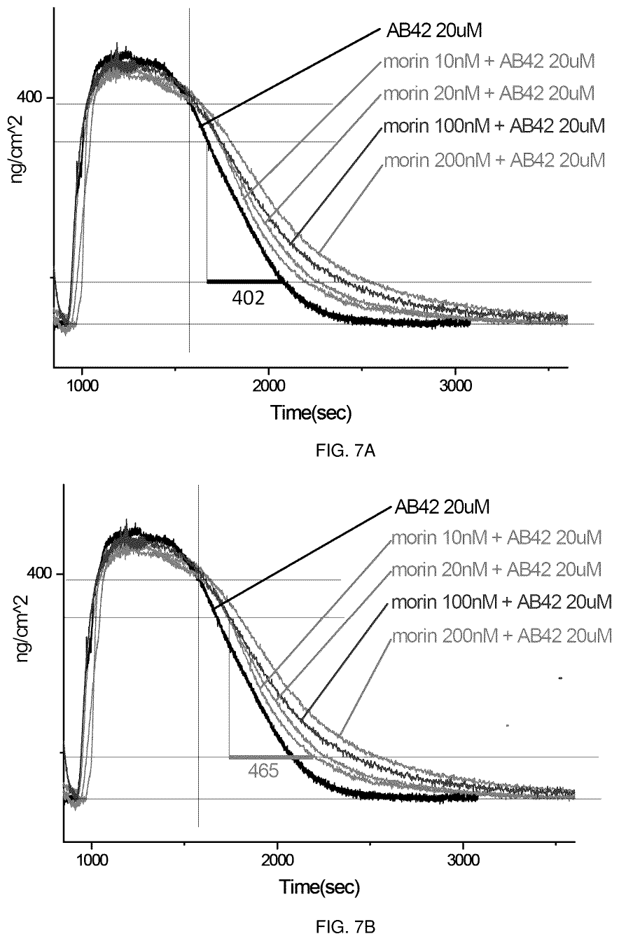

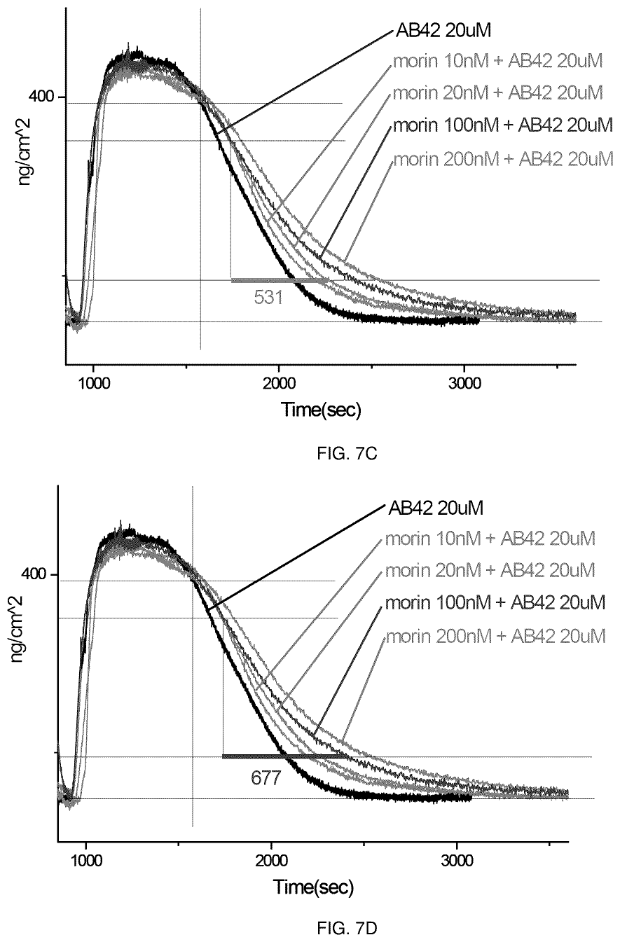

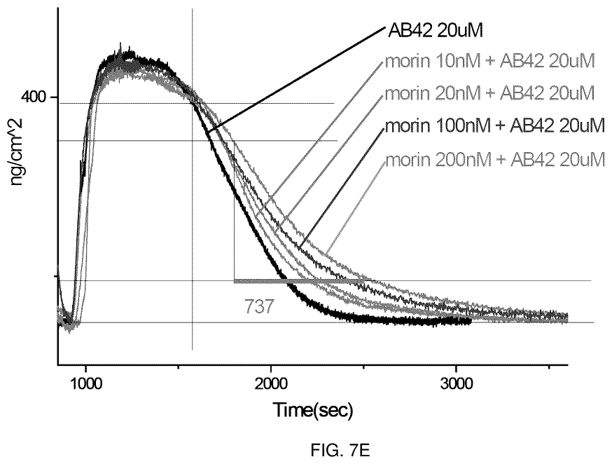

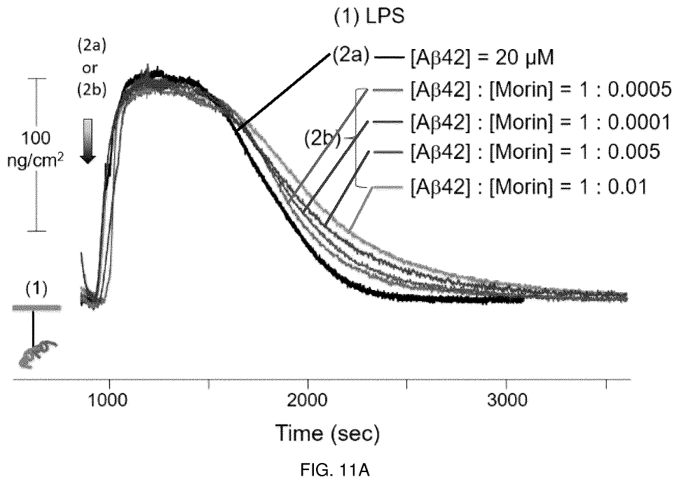

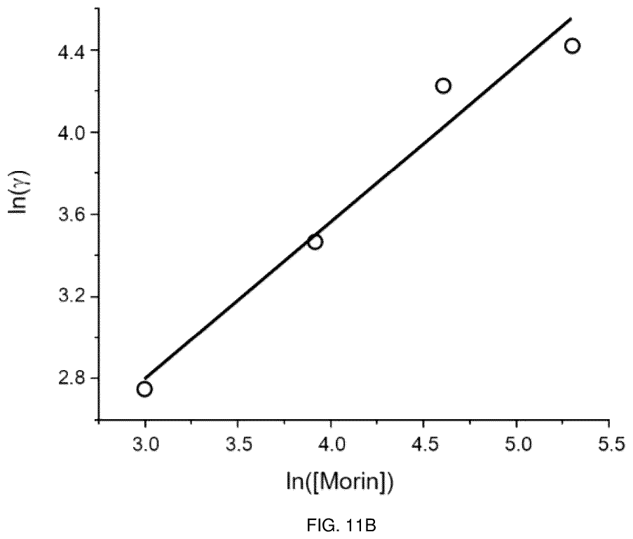

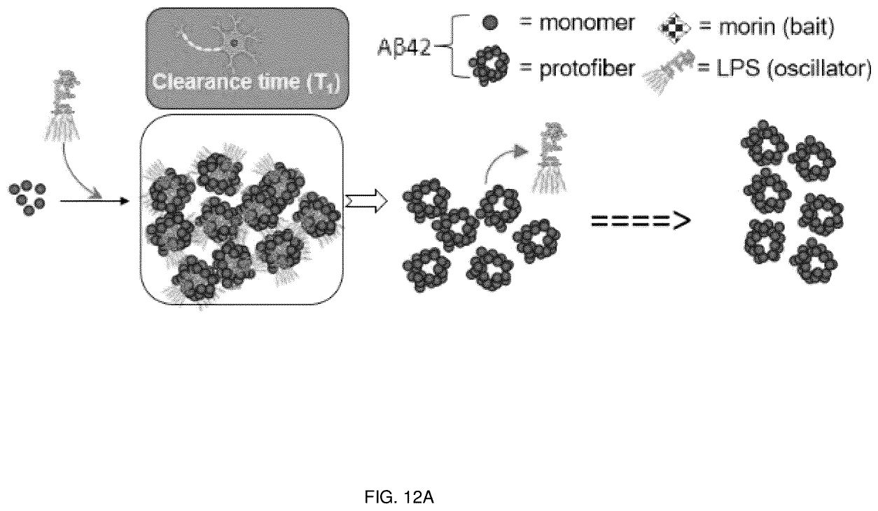

The intracellular degradation of Aβ42 protofibrils can be elicited by co-existence of LPS through endocytic clearance. Despite the relevance between the non-equilibrium behavior LPS-A342 and the efficiency of neuronal cells' Aβ42 clearance remaining unclear, we observed a significant time extension for the non-equilibrium interaction of Aβ42 in the LPS-morin combination (FIG. 11A). Thus, a bait-specific strategy may prolong the clearance time of neuronal cells because morin can sustain the non-equilibrium behavior of LPS-Aβ42 by intervening in the proceeding self-propagation of Aβ42. According to the experimental findings, the trend in the time expenditure from the up-to-down mass intensity was dose dependent in response to the ratios of Aβ42 to morin (FIG. 11A). The apparent lifetime increment of non-equilibrium oscillation under the co-existence of morin could still be treated as an indicator to evaluate the strength of non-equilibrium interaction between LPS and Aβ42. Analysis of the temporal profiles (FIG. 11A) is schematically presented in FIGS. 7A and 7B, and the relevant parameters are summarized in Table S1.

TABLE S1
The relevant parameters for constructing FIG. 11B.

| [morin] (nM) | time (s) | In([morin]) (s) | Δt (s)* | γ (%) ** | ln(γ) (%) |
|---|---|---|---|---|---|
| 0 | 402 | — | 0 | 0 | — |
| 20 | 465 | 2.996 | 63 | 15.7 | 2.752 |
| 50 | 531 | 3.912 | 129 | 32.1 | 3.469 |
| 100 | 677 | 4.605 | 275 | 68.4 | 4.225 |
| 200 | 737 | 5.298 | 335 | 83.3 | 4.423 |

*Δt = t_{w/morin} − t_{w/o morin}
${}^{**} γ = \frac{Δ t}{{t}_{w / o morin}} \times 100 \%$

An empirical relationship between the bait concentration and lifetime increment percentage, where this relationship was denoted γ, constructed to compare the binding strength of the LPS and Aβ42:

$$ℽ = {{ℽ}_{0} [ bait ]}^{n} \qquad ( 1 )$$

where γ₀ is a proportional constant and n is the power to which bait concentration is taken, conceptually referring to the interactive strength difference of non-equilibrium LPS-Aβ42 interaction. Here, morin represented an effective bait. Upon taking the natural log of Equation (1), n can be determined from the slope of ln(γ) versus In([morin]),

$$ln ( ℽ ) = ln ( {ℽ}_{0} ) + n ln ( [ morin ] ) \qquad ( 2 )$$

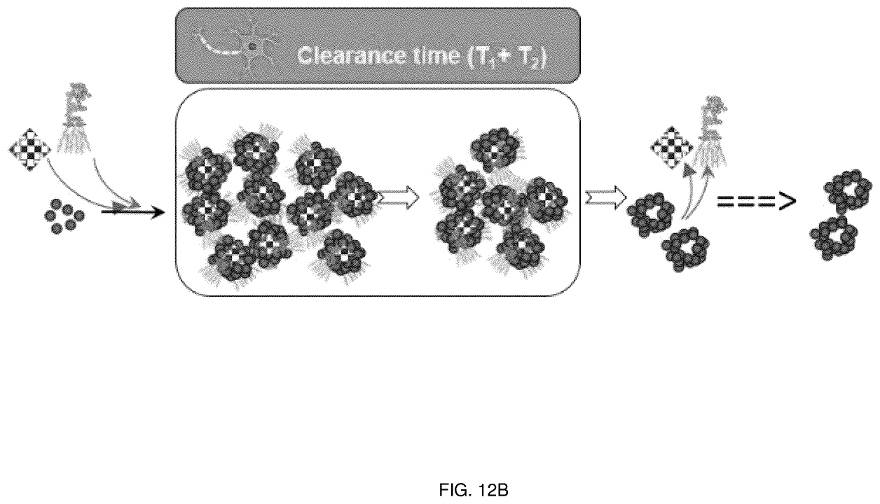

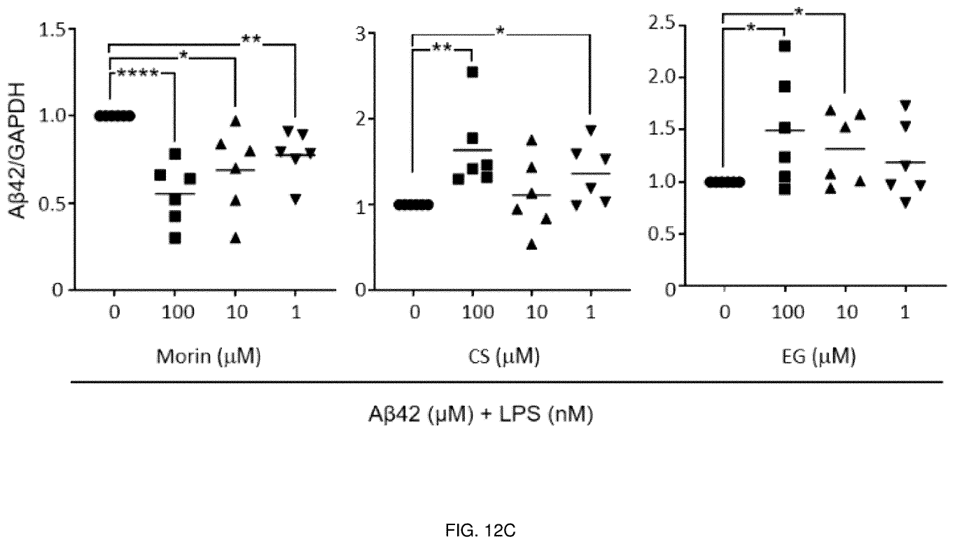

The relationship of ln(γ) with ln([morin]) manifested a satisfying linearity (FIG. 11B), with the value of n being 0.76±0.10. This value could be used to estimate the potential of trial drugs in comparison with morin. A high n refers to a more potent lengthening effect of the bait on the lifetime for the no-equilibrium oscillation, which may also imply a better efficiency of clearance by neural cells. Thus, we hypothesized that prolonging the non-equilibrium interaction between LPS and Aβ42 could act as a drug-predicting indicator for screening. This concept is illustrated in FIG. 12A. Accordingly, prolonging the non-equilibrium behavior of LPS-Aβ42 might be equivalent to extending the clearance time from T₁ (FIG. 12A) to T₁ plus T₂ (FIG. 12B) for the cargo uptake and clearance of neuronal cells from the cytosolic pools. In Western blot and statistical analyses (FIG. 12C), the residual Aβ42 levels from the total lysate were discovered to be significantly decreased after co-treatment with LPS and morin. The effect was not observed after co-treatment with LPS and CS or with LPS and EG. Accordingly, the clearance of Aβ42 strongly affected the survival of neuronal cells. Programming non-equilibrium oscillation may be a potential predictor for the screening of AD drugs.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

1. A method for selecting a drug candidate for treating or preventing or delaying the onset or progression of an amyloidogenic disease, comprising:
(a) contacting an analyte with amyloid peptide 1-42 (Aβ42) and an amphiphilic liposaccharide or a part thereof, wherein the amphiphilic liposaccharide or the part thereof is capable of forming a complex with Aβ42; and
(b) determining if the analyte extends an oscillatory event of the complex; and the analyte is selected as the drug candidate.

2. The method of claim 1, the drug candidate is for clearance of Aβ42.

3. The method of claim 1, wherein the amphiphilic liposaccharide comprises a lipid A part and an O-antigen part.

4. The method of claim 1, wherein the amphiphilic liposaccharide is selected from the group consisting of lipopolysaccharide (LPS), monophosphoryl lipid A (MPL), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-1-O-phosphono-α-D-glucopyranose (PIX), 2-deoxy-6-O-(2-deoxy-3-O-[(3R)-3-hydroxytetradecanoyl]-2-{[(3R)-3-hydroxytetradecanoyl]amino}-4-O-phosphono-β-D-glucopyranosyl)-3-O-[(3R)-3-hydroxytetradecanoyl]-2-{[(3R)-3-hydroxytetradecanoyl]amino}-D-glucopyranose (PXI) and salts thereof.

5. The method of claim 3, wherein step (a) comprises contacting the analyte with Aβ42 and the lipid A part or the O-antigen part of the amphiphilic liposaccharide.

6. The method of claim 1, wherein step (b) comprises observing the oscillatory events of the complex in the absence and presence of the analyte.

7. The method of claim 6, wherein step (b) comprises analyzing variation of mass signals over time to observe the oscillatory event of the complex.

8. The method of claim 7, wherein step (b) further comprises acquiring frequency signals and converting the frequency signals to the mass signals.

9. The method of claim 1, wherein step (b) comprises:
(b1) obtaining a first lifetime of the oscillatory event of the complex in the absence of the analyte;
(b2) obtaining a second lifetime of the oscillatory event of the complex in the presence of the analyte;
(b3) comparing the first lifetime and the second lifetime to determine if the analyte extends the oscillatory event of the complex.

10. A system for selecting a drug candidate for treating or preventing or delaying the onset or progression of an amyloidogenic disease, comprising:
Aβ42;
an amphiphilic liposaccharide or a part thereof, wherein the amphiphilic liposaccharide or a part thereof is capable of forming a complex with Aβ42; and
an apparatus for observing an oscillatory event of the complex.

11. The system of claim 10, wherein the amphiphilic liposaccharide comprises a lipid A part and an O-antigen part.

12. The system of claim 10, wherein the amphiphilic liposaccharide is selected from the group consisting of LPS, MPL, PIX, PXI and salts thereof.

13. The system of claim 11, wherein the amphiphilic liposaccharide or the part thereof is anchored on a sensor of the apparatus.

14. The system of claim 13, wherein the amphiphilic liposaccharide or the part thereof is anchored on the sensor of the apparatus by 3-mercaptopropanyln-hydroxysuccinimide ester.

15. The system of claim 13, wherein the apparatus comprises a mass-sensitive sensor.

16. The system of claim 15, wherein the apparatus comprises a quartz crystal microbalance.

17. The system of claim 15, wherein the apparatus acquires frequency signals and converts the frequency signals to mass signals.

18. A method for treating or preventing or delaying the onset or progression of an amyloidogenic disease in a subject in need, comprising administering a therapeutically effective amount of morin and the amphiphilic liposaccharide or a part thereof or a pharmaceutical composition comprising the same to the subject, wherein the morin and the amphiphilic liposaccharide or a part thereof are co-administered simultaneously, separately or sequentially or co-administered in combination as a coformulation, and the amphiphilic liposaccharide or a part thereof is capable of forming a complex with Aβ42.

19. The method of claim 18, wherein the amyloidogenic disease is selected from the group consisting of Alzheimer's disease, mild cognitive impairment, Parkinson's disease with dementia, Down's syndrome, diffuse Lewy body disease, cerebral amyloid angiopathy, vascular dementia, and mixed dementia.

20. The method of claim 18, which is further for clearance of Aβ42 in a subject in need.
